SEQUENCE LISTING (38 sequences)

SEQ ID NO: 1 - protein, 54 aa, hepatitis C virus
  Peptide (1)..(54): This sequence is shown in the specification using one-letter codes
STWVLVGGVLAALAAYCLSTGCVVIVGRIVLSGKPAIIPDREVLYQEFDEMEEC

SEQ ID NO: 2 - protein, 12 aa, Artificial sequence
  Binding and activation domain
CVVIVGRIVLSG

SEQ ID NO: 3 - protein, 14 aa, Artificial sequence
  Native amino acid sequence of NS4A′s binding and activation domain
GSVVIVGRIVLSGK

SEQ ID NO: 4 - protein, 6 aa, Artificial sequence
  Consensus sequence
DEMEEX

SEQ ID NO: 5 - protein, 4 aa, Artificial sequence
  Consensus sequence
LVIV

SEQ ID NO: 6 - protein, 4 aa, Artificial sequence
  Consensus sequence
CVIV

SEQ ID NO: 7 - protein, 4 aa, Artificial sequence
  Consensus sequence
FVIV

SEQ ID NO: 8 - protein, 7 aa, Artificial sequence
  Consensus sequence
VVIVGRI

SEQ ID NO: 9 - protein, 8 aa, Artificial sequence
  Consensus sequence
VIVGRIVL

SEQ ID NO: 10 - protein, 7 aa, Artificial sequence
  Consensus sequence
IVGRIVL

SEQ ID NO: 11 - protein, 6 aa, Artificial sequence
  Consensus sequence
VGRIVL

SEQ ID NO: 12 - protein, 5 aa, Artificial sequence
  Consensus sequence
GRIVL

SEQ ID NO: 13 - protein, 13 aa, Artificial sequence
  Consensus sequence
DEMEEXXXXXXXX

SEQ ID NO: 14 - protein, 6 aa, Artificial sequence
  Consensus sequence
DEMEED

SEQ ID NO: 15 - protein, 6 aa, Artificial sequence
  Consensus sequence
DEMEEE

SEQ ID NO: 16 - protein, 9 aa, Artificial sequence
  Binding sequence of the domain of NS4A
VVIVGRIVL

SEQ ID NO: 17 - protein, 6 aa, Artificial sequence
  Sequence of the NS4A/5B junction
DEMEEC

SEQ ID NO: 18 - protein, 6 aa, Artificial sequence
  Sequence for the P1 position
DEMEEX

SEQ ID NO: 19 - protein, 16 aa, Artificial sequence
  binding and activation domain
KKGSVVIVGRIVLSGK

SEQ ID NO: 20 - protein, 9 aa, Artificial sequence
  binding and activation sequence as described in the specification
VVIXGRIVL

SEQ ID NO: 21 - protein, 12 aa, Artificial sequence
  binding and activation domain
VVIVGRIVLSGK

SEQ ID NO: 22 - protein, 11 aa, Artificial sequence
  binding and activation domain
GCVVIVGRIVL

SEQ ID NO: 23 - protein, 4 aa, Artificial sequence
  Domain in NS4A that binds to NS3 as discussed in the specification (e.g., page 37)
LSGK

SEQ ID NO: 24 - protein, 4 aa, Artificial sequence
  Domain in NS4A that binds to NS3 as discussed in the specification (e.g., page 37)
GCVV

SEQ ID NO: 25 - protein, 5 aa, Artificial sequence
  Domain in NS4A that binds to NS3 as discussed in the specification (e.,g page 37)
VLSGK

SEQ ID NO: 26 - protein, 5 aa, Artificial sequence
  Domain in NS4A that binds to NS3 as discussed in the specification (e.g., page 37)
GCVVI

SEQ ID NO: 27 - protein, 6 aa, Artificial sequence
  Domain in NS4A that binds to NS3 as discussed in the specification (e.g., page 37)
IVLSGK

SEQ ID NO: 28 - protein, 6 aa, Artificial sequence
  Domain in NS4A that binds to NS3 as discussed in the specification (e.g., page 37)
GCVVIV

SEQ ID NO: 29 - protein, 7 aa, Artificial sequence
  Domain in NS4A that binds to NS3 as discussed in the specification (e.g., page 37)
RIVLSGK

SEQ ID NO: 30 - protein, 7 aa, Artificial sequence
  Domain in NS4A that binds to NS3 as discussed in the specification (e.g., page 37)
GCVVIVG

SEQ ID NO: 31 - protein, 8 aa, Artificial sequence
  Domain in NS4A that binds to NS3 as discussed in the specification (e.g., page 37)
GRIVLSGK

SEQ ID NO: 32 - protein, 8 aa, Artificial sequence
  Domain in NS4A that binds to NS3 as discussed in the specification (e.g., page 37)
GCVVIVGR

SEQ ID NO: 33 - protein, 9 aa, Artificial sequence
  Domain in NS4A that binds to NS3 as discussed in the specification (e.g., page 37)
VGRIVLSGK

SEQ ID NO: 34 - protein, 9 aa, Artificial sequence
  Domain in NS4A that binds to NS3 as discussed in the specification (e.g., page 37)
GCVVIVGRI

SEQ ID NO: 35 - protein, 10 aa, Artificial sequence
  Domain in NS4A that binds to NS3 as discussed in the specification (e.g., page 37)
IVGRIVLSGK

SEQ ID NO: 36 - protein, 10 aa, Artificial sequence
  Domain in NS4A that binds to NS3 as discussed in the specification (e.g., page 37)
GCVVIVGRIV

SEQ ID NO: 37 - protein, 11 aa, Artificial sequence
  Domain in NS4A that binds to NS3 as discussed in the specification (e.g., page 37)
VIVGRIVLSGK

SEQ ID NO: 38 - protein, 9 aa, Artificial sequence
  Amino acid sequence from a library as described in Figure 10 and page 39 of the specification
VVIVXRIVL

(19) United States
(12) Patent Application Publication
(10) Pub. No.: US 2003/0176689 A1
(43) Pub. Date: Sep. 18, 2003

(54) Inhibitors of hepatitis C virus protease

(76) Inventors: Michael Joyce, Edmonton (CA); Mark Williams, Edmonton (CA); Ole Hindsgaul, Edmonton (CA); D. Lorne Tyrrell, Edmonton (CA)

Correspondence Address: BOZICEVIC, FIELD & FRANCIS LLP, 200 MIDDLEFIELD RD, SUITE 200, MENLO PARK, CA, 94025, US

(21) Appl. No.: 10/319,402

(22) Filed: Dec. 13, 2002

Related U.S. Application Data
(60) Provisional application No. 60/340,574, filed on Dec. 14, 2001.

(51) Int. Cl. A61K 38/08; C07K 7/06; A61K 38/00; C07K 5/00; A61K 38/04; C07K 7/00; C07K 16/00; C07K 17/00; C07H 21/04

(52) U.S. Cl. 536/24.5; 530/329; 530/328; 514/16; 514/17

(57) ABSTRACT

The present invention features HCV protease inhibitors, which act by affecting the activity of the HCV protease NS3, or by preventing its activation by NS4A. The invention also features methods of use of the inhibitors of the invention in the treatment of HCV infection in a subject.

### CROSS-REFERENCE TO RELATED APPLICATIONS

This application claims priority benefit to U.S. provisional application serial No. 60/340,574, filed Dec. 14, 2001, which application is incorporated herein by reference in its entirety.

### GOVERNMENT SUPPORT

This invention was supported at least in part by a U.S. grant from the National Institute of Allergy and Infectious Disease, grant no. IR43AI048347/01A1. The U.S. government may have certain rights in this invention.

### FIELD OF THE INVENTION

The invention generally relates to Hepatitis C virus (HCV) protease inhibitors, and their use in treatment of HCV infection.

### BACKGROUND OF THE INVENTION

Hepatitis C Virus (HCV) is a global health problem with estimates of more than 1% of the world's population currently infected with the virus (Alter Hepatology 1997; 26:635-655). One of the truly outstanding characteristics of HCV is its ability to establish chronic infections in 65-80% of infected patients (Alter et al. NEJM 1999:340:556-562). Chronic infection with HCV can lead to serious sequellae including chronic active hepatitis, cirrhosis and hepatocellular carcinoma—usually manifest 10, 20 and 25 years respectively after the initial infection (Alter et al. NEJM 1992; 222:1899-1902; Iwarson et al. Clin. Infect. Dis. 1995; 20:1361-1370). End stage liver disease from HCV has become the leading indication for liver transplantation in North America. It has been suggested that there will be a 2-3 fold increase in liver transplantation in 10 years as a result of cirrhosis from hepatitis C.

Discovered in 1989 (Choo et al. Science 1989; 244:359-362), the virus, classified as a Flavivirus, has a 9.5 kilobase positive-strand RNA genome which encodes a single polypeptide of 3008-3037 amino acids long (Clarke J. Gen. Virol. 1997;78:2397-2410). The virus exhibits nucleotide sequence variation within and between geographic regions of the world. European and North American isolates are more closely related to each other than Japanese isolates. Based on the genetic variability of the virus, which can be up to 30% at the nucleotide level, at least 6 genotypes and more than 30 subtypes have been identified (Bukh et al. Seminars in Liver Disease. 1995; 15:41-63; Simmonds Hepatology. 1995; 21:570-583; Simmonds et al. Hepatology. 1994;19:1321-1324). This variability has implications for vaccine and antiviral drug development. At present the only approved therapies are interferon, with or without ribavirin, which is only successful in a small portion of patients. There is therefore an urgent need to develop novel antivirals to treat HCV (Reichard et al. Hepatology, 1997; 3 suppl. 1:108S-111S).

Many components of the HCV polyprotein and genome have been identified and characterized. The open reading frame (ORF) of HCV is flanked by a non-translated region (NTR) of 341 nucleotides at the 5′ end, and approximately 200 nucleotides at the 3′ end containing a poly-U tract (negative strand) and a highly conserved 98 base sequence (Tanaka et al. J. Virol. 1995; 70:3307-3312; Kolykhalor et al. J. Virol. 1996;70:3363-3371; Yamada et al. Virology. 1996; 223:255-261). The 5′ NTR is also highly conserved between species and has been proposed as a major target for antisense technologies (e.g. ribozymes) owing to this conservation. It contains a structure similar to the 5′ NTR of picornaviruses which serves as an internal ribosome entry site (IRES). This highly conserved sequence forms a stable hairpin structure and is required for cap-independent translation both in vitro and in vivo (Honda et al. RNA. 1996 October;2(10):955-68).

The 3′ UTR contains a tripartite structure with a conventional 3′ end (approximately 30 nucleotides), a poly-U tract (variable length) and a highly conserved 98 nucleotide 3′X tail (Major et al. Hepatology. 1997;25:1527-1538). This 3′X tail is similar in structures seen in other RNA viruses and is important for RNA synthesis and genome packaging.

The core protein located at the N-terminal end of the ORF is the viral capsid protein. The core protein is released from the viral polypeptide by host proteases (Hijikata et al. PNAS. 1991; S&5547-5551). In addition to binding to viral RNA, the core protein has also been shown to suppress apoptotic cell death (Roy et al. Virology. 1996, 176:182-185).

The envelope proteins E1/E2 are the major glycoproteins on the surface of the virus. They are released from the viral polypeptide by host-cell peptidases. Both envelope proteins are heavily glycosylated, and as such, glucosidase inhibitors have been proposed as potential antivirals. E1 (gp 35) protein, through its carboxy terminus, can associate with the core protein (Lo et al. J. Virol. 1996; 70:5177-5182). The E2 (gp 70) protein can associate with p7, an HCV protein of unknown function, however its more important interaction may be with E1.. E1 and E2 have been shown to form intracellular aggregates linked by inter and intra-molecular disulphide bonds as well as non-covalent complexes (Lanford et al. Virology. 1993; 122:225-235; Grakoui A, et al. J. Virol. 1993; 67:1385-1395). The envelope proteins are possible candidates for prophylactic vaccine development, but E2 possesses an N-terminal 34 amino acid hyper-variable region which mutates quickly and may escape neutralizing antibodies. This in turn could contribute to the persistence of infections.

The HCV polyprotein is cleaved by a mixture of host and viral proteases (Major et al. 2001 Fields Virology (Knipe and Howley ed.) Vol. 1, 4th ed., pp1127-1161). Lippincott Williams and Wilkins). The NS2 gene encodes a zinc2+metalloprotease. This enzyme requires both the carboxy terminus of NS2 and the amino terminus of NS3 for its activity. This enzyme produces a cis-cleavage between NS2 and NS3 (Reed et al. J. Virol. 1995;69:4127-4136). The cleavage of the NS2/3 junction releases the N-terminus of NS3, the serine protease responsible for the majority of the polypeptide cleavages. The NS2/3 junction is potential site of inhibitor development but most of the attention has been focused on NS3 as a target for protease intervention.

The NS3 gene codes for a protein of approximately 70 kDA. The protein is a serine protease which is responsible for the proteolytic processing downstream from NS3 (as indicated by arrows from NS3 in FIG. 1). In general, NS3 is responsible for cleavage of non-structural proteins. (Hijikata, M., et al 1993 J. Virol 67:4665-46675). The protease activity resides in the N-terminal third of the protein; the remaining two-thirds of NS3 have NTPase and helicase activity. As discussed in more detail below, NS3 requires NS4A as a cofactor for some cleavage sites (Bartenschlager et al. J. Virol. 1993; 67:3835-3844; Tomei et al. J. Virol. 1993; 67:4017-4026; Bartenschlager et al. J. Virol-1995; 69:7519-7528). The crystal structure of the active complex of NS3/4A has been reported (Kim et al. Cell. 1996; 87: 343-355; Erratum in Cell Apr. 4, 1997; 89(1):159).

The NS4A gene encodes for NS4A and NS4B proteins. NS4A, a cofactor for the NS3 protease, contains a highly conserved central domain that is responsible for the activation of NS3. The N-terminus encodes a 20 amino acid region that is believed to form a transmembrane domain which thereby anchors NS3 to the endoplasmic reticulum membrane (Wight-Minogue et al. J. Hepatol 2000; 32:497-504).

The NS5 gene encodes NS5A and 5B proteins. NS5A is a phosphorylated protein, but its functional role in the replicative cycle remains unclear. The mutations in NS5A have been found to be correlated with decreased sensitivity to interferon (Enomoto et al. J. Clin. Invest. 1995; 96:224-230; Enomoto et al. NEJM. 1996; 334:77-81). The NS5B protein is highly conserved between strains and between flaviviruses and pestiviruses. The NS5B protein contains an amino acid motif G-D-D which is characteristic of all known RNA-dependent RNA polymerases. NS5B is also seen as a potential target for small molecule intervention and the development of antivirals (Behrens et al. EMBO J. 1996; 15:12-22).

The first cleavage that the NS3 protease performs liberates the NS4A protein (Bartenschlager et al. 1994 J.Virol. 68:5045-5055). The NS4A protein is 54 residues long (stwvlvggvl aalaayclst gcvvivgriv lsgkpaiipd revlyqefde meec; see, e.g., GenBank Accession No. NP_{—}751925. and activates the NS3 protease (see, e.g., Koch et al. Virology 1996 221(1):54-66; Gallinari et al. Biochemistry 1999; 38(17):5620-32). NS4A is thought to also be responsible for NS3 membrane localization (Bartenschlager et al. 1994 J.Virol. 68:5045-5055). The binding and activation domain of NS4A is contained in a conserved 14 residue sequence found in the central part of the NS4A protein. A 12-amino-acid peptide containing amino acid residues 22 to 33 in NS4A (CVVIVGRIVLSG) has been reported to contain the binding and activation domain, as it is both sufficient and necessary for cofactor activity in NS3-mediated cleavage at NS4A/4B, and enhances NS3-mediated cleavage at the NS5A/5B site (Butkiewicz et al. Virology 1996 225(2):328-38).. Interaction of NS4A with NS3 lowers the Km of NS3 for substrate by 5-8 times and raises the Kcat by 3-16 times (Landro, J A., et al. 1997 Biochem. 36:9340-9348). The crystal structure of NS3 bound to NS4A peptides containing the binding and activation domain has been described (Yan et al. Protein Sci 1998; 7(4):837-47; Love et al. Clin Diagn Virol 1998; 10(2-3):151-6). Expression of a recombinant NS3-4A proteinase fusion protein in Escherichia coli and the in vitro characterization of NS3 enzyme activity using synthetic peptide substrates is described in Attwood et al. Antivir Chem Chemother 1999; 10(5):259-73.

Due to the success of protease inhibitors in treating human immunodeficiency virus (HIV) infection, the protease activity of the NS3 protein is one of the main targets for antiviral therapy in HCV (for reviews see, e.g., Kwong et al. Antiviral Res 1999; 41(1):67-84; Kwong et al. Antiviral Res 1998; 40(1-2):1-18). As noted above, the protease activity of NS3 is contained within the N-terminal one-third of the gene, and is responsible for cleavage of the non-structural proteins from the HCV polyprotein. The sites where protein cleavage occurs in a substrate can be represented by the following formula, with 6 amino acid residues (P) positioned N-terminal to the cleavage site, and 4 amino acids (P′) C-terminal to the cleavage site:

P6-P5-P4-P3-P2-P1-Δ-P1′-P2′-P3′-P4′

The consensus amino acid sequence for NS3 protease substrates can also be represented as a ten amino acid motif, which motif is provided below:

(D/E)-(D/E)-X1-X2-X3-(C/S)Δ(A/T)-X4-X5-X6

where X1, X2, X3, X4, X5, X6=any amino acid, C=cysteine, S=serine, A=alanine, D=aspartic acid, and E=glutamic acid. Several peptide analogs based on the amino acid sequence of the “P side” of the cleavage junction (based on the formula (D/E)-(D/E)-X1-X2-X3-(C/S)) have been described (LaPlante et al. J. Biol. Chem. 1999 274, 18618-24; U.S. Pat. Nos. 6,143,715; 6,187,905; Fattori et al, J. Biol. Chem. 2000 275:7152-7; Pessi et al. Biochem. 1998 37:8906-14; See also U.S. Pat. No. 5,866,684; Steinkuhler et al. Biochemistry 1998 37(25):8899-905; Llinas-Brunet et al. Bioorg Med Chem Lett 2000 10(20):2267-70); Bennett et al. Bioorg Med Chem Lett. Feb. 12, 2001; 11(3):355-7; Pessi et al. J Pept Sci 2001; 7(1):2-14; Ingallinella et al. Biochemistry. Apr. 30, 2002; 41(17):5483-92;Bianchi et al. Biopolymers 2002 66(2):101-14. Crystal structures of the serine protease domain of NS3 complexed with NS4A cofactor and with two potent, reversible covalent inhibitors spanning the P1-P4 residues have been described (Di Marco et al. J Biol Chem Mar. 10, 2000; 275(10):7152-7).

Another strategy for inhibiting NS3 protease activity is to identify peptide analogs based on the sequence of NS4A that interacts with NS3. The native amino acid sequence of NS4A's binding and activation domain is GSVVIVGRIVLSGK. Putative peptide analogs based on this binding and activation domain of NS4A are described in, for example, U.S. Pat. No. 5,990,276. Other NS3 protease inhibitors based on peptides derived from complementarity-determining regions (CDRs) of a monoclonal antibody have been reported (see, e.g., Tsumoto et al. FEBS Lett. Aug. 14, 2002; 525(1-3):77-82).

There is a continuing need in the field for antiviral compounds effective against HCV, including HCV protease inhibitors suitable for the use in treatment of HCV infection. The present invention addresses this need.

### SUMMARY OF THE INVENTION

The present invention features viral protease inhibitors, particularly HCV protease inhibitors. In one embodiment, the inhibitors of the invention are characterized by binding viral NS3 protease, preferably without significant or detectable activation of NS3 activity in cleaving the NS3/NS4A substrate. In an embodiment of particular interest, the peptides have the formula:

X₁-Z₁-Z₂-Z₃-Z₄-X₂

where X₁ is an integral number of residues from 0 to 5; Z₁ is a hydrophobic residue; Z₂ is a D-isomer of a hydrophobic residue; Z₃ is a positively charged residue; Z₄ is a hydrophobic residue or a positively charged residue; and X₂ is an integral number of residues from 0 to 3, wherein X₂ may optionally comprises a terminal amino group. The invention also features methods of use of these inhibitors in the treatment of HCV infection in a subject.

In one aspect, the invention features compositions which interfere with the protease activity of the HCV NS3 protein. Another aspect of the invention features compositions which inhibit the NS4A-enhanced activation of NS3. Yet another aspect of the invention features methods of treating diseases associated with Hepatitis C viral infection.

In certain embodiments, the inhibitors of the invention are agents that decrease the efficiency or stability of the association of the NS3 and NS4A subunits of the HCV protease. Such inhibitors include agents that bind NS3 so as to decrease the efficiency or prevent NS4A binding to NS3, and agents that are able to disrupt NS3-NS4A complexes. These inhibitors also include agents which prevent NS4A from increasing the protease activity of NS3.

In other embodiments, the inhibitors of the invention are agents that interfere and/or decrease substrate binding and/or cleavage by NS3. Such inhibitors are agents which bind to NS3 in the substrate binding site region and/or agents which interfere with substrate binding to NS3 as well as inhibiting substrate-NS3 complexes. The NS3 protease inhibitors of the present invention also include agents that interact with NS3 so as to decrease the efficiency or prevent the protease activity of NS3.

In another embodiment, the inhibitors of the invention are agents which interfere with both substrate binding and NS4A binding to NS3. Such inhibitors comprise molecules with a domain which decreases the efficiency or stability of the association of the NS3 and NS4A subunits as well as a domain which interferes with substrate binding to NS3 and inhibits substrate-NS3 complexes. The domains of the inhibitor agent are covalently linked, directly or via a linker.

In one aspect the invention features a peptide agent having the formula:

X₁-Z₁-Z₂-Z₃-Z₄-X₂

where X₁ is an integral number of residues from 0 to 5, Z₁ is a hydrophobic residue, preferably a large hydrophobic residue, Z₂ is a D-isomer of a hydrophobic residue, preferably a D-isomer of a large hydrophobic residue, Z₃ is a positively charged residue, preferably a large positively charged residue; Z₄ is a hydrophobic residue or a positively charged residue; and X₂ is an integral number of residues from 0 to 3, wherein X₂ may optionally comprises a terminal amino group. In specific embodiments, Z₁ is M, F, W, or Cha, preferably Cha; Z₂ is a D-amino acid of v, a, l, i, p, m, f, w, or chA; Z₃ is K, R, or H, preferably K or R; and Z₄ is any of Z₁ or Z₃. References throughout the specification to characteristics of residues, such as charge or hydrophobicity, refer to these characteristics at about pH 7.0.

In a specific embodiment of interest, the peptide has the formula:

X₁-Cha-v-(R/K)-(Cha/R)-X₂

where X₁ is an integral number of residues from 0 to 5, X₂ is an integral number of residues from 0 to 3, Cha is cyclohexylalanine, v is D-valine, and wherein X₂ optionally comprises a terminal amino group.

In further embodiments, X₁ is W, R, FFR, FNW, FNR, or FFW. In further embodiments, X₂ is a carboxyl group or I-Cha. In still other embodiments, Cha is β-cyclohexylalanine. In still other embodiments, the peptide has the formula W-Cha-v-R-Cha-I-Cha; R-Cha-v-R-Cha-I-Cha; F-F-W-Cha-v-R; W(Cha)vR(Cha)I(Cha)-NH2; R(Cha)vR(Cha)I(Cha)-NH2; FFW(Cha)vR(Cha)-NH2; or R-Cha-v-R-Cha-I-Cha.

In another aspect, the invention features a peptide having the formula:

X₁-GRI-X₂

where X₁ represents an integral number of residues from 0 to 9, and X₂ represents an integral number of residues from 0 to 10, with the proviso that the peptide is less than 13 residues. In specific embodiments, any of G, R, or I is a D-amino acid, with substitution of G by a D-amino acid being of particular interest. In other embodiments, X₁ is VV, VIV, IV, V, LVIV, t-LVIV, CVIV, PenVIV, FVIV, or an amino group. In still other embodiments, X₁ is C, Q,E, F, ChA, W, R, or Y, or a D isomer thereof. In further embodiments, X₂ is a carboxyl group, V, VL, D-val, (D-val)L, V(D-leu) or (D-val)(D-leu). In a specific embodiment, the peptide is of the formula VVIVGRI, VIVGRIVL, IVGRIVL, VGRIVL or GRIVL, where one or more of the residues of the peptide are a D-amino acid.

In yet another aspect, the invention features a peptide having the formula:

X₁-DEMEE-X₂

wherein X₁ represents an acetyl group or an integral number of residues from 0 to 7, and X₂ is an acidic amino acid, with the proviso that the peptide is less than 13 residues in length. In specific embodiments, X₂ is Asp or Glu.

In a further specific embodiments, the peptide has the formula:

X₁-DEMEEX₂-X₃

wherein X₁ represents an acetyl group or an integral number of residues from 0 to 7, and X₂ is an acidic amino acid, and X₃ represents an integral number of residues from 0 to 7, with the proviso that the peptide is less than 13 residues in length. In other embodiments, the peptide has a formula selected from the group consisting of DEMEED, DEMEEE, Ac-DEMEED-OH and Ac-DEMEEE-OH.

In another aspect the invention features a compound having the formula:

Peptide₁-L-Peptide₂

where each of Peptide, and Peptide₂ is a peptide as described above, and L represents a linker that links Peptides 1 and 2. In a specific embodiment, the linker is an amino acid sequence comprising polar amino acid residues, with a linker comprising D-arginine being of particular interest.

In related aspects, the invention features a pharmaceutical composition comprising a peptide or compound of the invention.

In further related aspects, the invention features methods for inhibiting replication of hepatitis C virus (HCV) and/or reducing viral load, in a subject having an HCV infection by administering to the subject a peptide or compound of the invention in an amount effective to inhibit HCV replication and/or reduce viral load in the subject.

The invention thus provides HCV inhibitors that bind NS3 without substantial or detectable activation of NS3 activity by binding of NS4A.

The invention also provides, in certain embodiments, inhibitors that bind NS3 without activation of the protease above NS3 activity in the absence of NS4A, and preferably decrease NS3 activity relative to NS3 activity in the absence of NS4A.

The inhibitors of the invention can also inhibit other activities that involve or require NS4A binding, or which are inhibited by native NS4A. For example, NS2/3 autocleavage, which is decreased in the presence of NS4A, can be inhibited using certain inhibitors of the invention that are NS4A mimics (i.e., contain a motif that mimics NS4A so as to bind an NS4A-binding site of a protein).

An advantage of the invention is that the inhibitors are small hydrophobic peptides, which are more amenable to crossing the cellular membrane of an HCV-infected host cell.

These and other advantages, and features of the invention will become apparent to those persons skilled in the art upon reading the details of the NS3 protease inhibitor compositions and methods of use are more fully described below.

### BRIEF DESCRIPTION OF THE DRAWINGS

FIG. 1 is a diagram of the polyprotein processing of HCV.

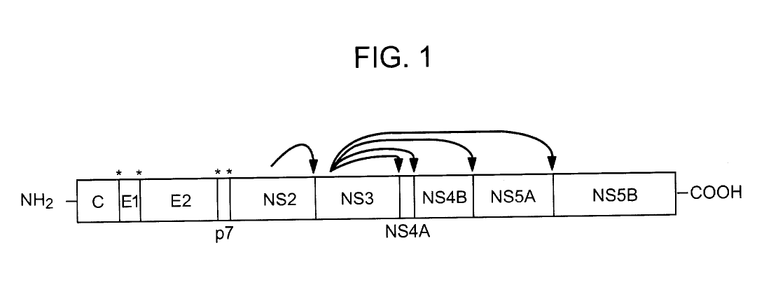

FIG. 2 is a FAC-MS readout showing the NS3 binding activity of peptide Ac-DEMEED-OH.

FIG. 3 is a graph of the elution time for a peptide which inhibits HCV NS3 protease.

FIG. 4 is a graph showing the binding of Val23 substituted NS4A peptides to NS3 as indicated by elution profiles using FAC-MS.

FIG. 5 is a graph showing the relative binding of Val26 substituted NS4A minimal binding peptides to NS3.

FIG. 6, which includes Panels A and B, are graphs showing binding of the N-terminal deletion library to an NS3 affinity column and analysis by FAC-MS.

FIG. 7, which includes Panels A and B, are graphs showing binding of the C-terminal deletion library to an NS3 affinity column and analysis by FAC-MS.

FIG. 8 is a graph showing the correlation between change in elution time of NS4A deletion peptides and buried surface area of each deleted residue.

FIG. 9 is a graph showing the binding of short (minimal binding) NS4A peptides to an NS3 affinity column, with analysis of FAC-MS.

FIG. 10 is a graph showing an exemplary chromatogram from a minimal binding NS4A peptide library with changes at residue glycine 27.

FIG. 11 is a table summarizing the results of FAC-MS analysis of various minimal binding NS4A mutant peptides.

FIG. 12 is a graph showing inhibition of cleavage of 5A-pNA by NS3 and wildtype NS4A by various mutant NS4A peptides.

FIG. 13 is a graph showing the IC50 of Peptide 3 from FIG. 13.

FIG. 14 is a photograph showing inhibition of NS3 levels in a culture of T and B cells by varying concentration of Peptides 1, 3, or 5 from FIG. 13.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a peptide” includes a plurality of such peptides and reference to “the inhibitor” includes reference to one or more inhibitors and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

By “isolated” is meant that the, for example, peptide of the invention is at least 60%, by weight, free from the proteins and naturally-occurring organic molecules with which it is naturally associated or, in the context of synthetic peptides, at least 60% by weight free of synthetic peptides of different sequence and intermediates. Preferably, the preparation is at least 75%, more preferably at least 90%, and most preferably at least 99%, by weight, peptide. An isolated peptide as described herein may be obtained, for example, by chemically synthesizing the protein or peptide, or by expression of a recombinant nucleic acid encoding a peptide of interest, with chemical synthesis likely being preferred. Purity can be measured by any appropriate method, e.g., column chromatography, mass spectrometry, HPLC analysis, and the like.

The term “peptide” refers to a polymeric form of amino acid residues (including amino acid residue derivatives) of any length (e.g., at least two, usually at least 3 to 4, more usually at least 5 to 6, often about 6, generally 7 to 8, normally about 9 residues, generally not more than 14, usually less than 12, more usually less than 10 residues in length), which can include coded and non-coded amino acids, chemically or biochemically modified (e.g., post-translational modification such as glycosylation) or derivatized amino acids, polymeric polypeptides, and polypeptides having modified peptide backbones. The natural amino acids may be selected from alanine (A), arginine (R), asparagine (N), aspartic acid (D), cysteine (C), glutamine (Q), glutamic acid (E), glycine (G), histidine (H), isoleucine (I), leucine (L), , lysine (K), methionine (M), phenylalanine (F), proline (P), serine (S), threonine (T), tryptophan (W), tyrosine (Y), and valine (V). A pseudo-amino acid includes an amino acid wherein the amino or carbonyl function has been replaced by another chemical group.

Exemplary residues that can be incorporated into the peptides of the invention include, without limitation, cyclohexylalanine (“Cha”, α- or β-cyclohexylalanine, preferably β-cyclohexylalanine), penacillamine (“Pen”), acetylated amino acid residues (“Ac”), and amino acid residues modified by an alkyl groups, e.g., a tert-butyl group (“tert” or “t”), nitrophenylalanine, homoarginine, thiazolidine, aminobutyric acid (Aib), aminoisobutyric acid (Abu), and the like. Other non-coded amino acids of interest may include: L-α-aminobutyric acid (Abu), 2-aminoisobutyric acid (Aib), γ-aminobutyric acid (γAbu), 6-aminohexanoic acid (Ahx), 1-amino-1-cyclohexanecarboxylic acid (Acx), homophenylalanine (Hof), norvaline (Nva), omithine (Orn), 4-chlorophenylalanine (Fcl), 4-nitrophenylalanine (Fno), phenylglycine (Phg), (3S,4S)-4-amino-3-hydroxy-5-cyclohexylpentanoic acid (Ach), (3S,4S)-4-amino-3-hydroxy-5-phenylpentanoic acid (Ahp), 5-amino valeric acid (Ava), 8-aminooctanoic acid (Aoc), β-diaminopropionic acid (βDP), α-diaminopropionic acid (αDP), (3S,4S)-4-amino-3-hydroxy-6-methylheptanoic acid (Sta), 1,2,3,4-tetrahydroisoquinoline-3-L-carboxylic acid (Tic). 3,3-diphenlyalanine (Dif), 3-pyridylalanine (Pyr), 2-naphthylalanine (Nap), 4-thiazolylalanine (Thz), 4-fluorophenylalanine (Pff), and 4-carboxymethylpiperazine (Cmpi). Residues may be in L- or D-isomer form, where such is available. These residues may be incorporated into the peptides of the invention given the guidance herein regarding the size and character (e.g., hydrophobicity, charged, etc.) of residues in the peptides of the invention.

The sequence of the peptides described here is described by the following nomenclature: the wild-type residue modified, the position of the residue in the wildtype NS4A polypeptide, and the residue substituted at that position. Thus, for example V23F indicates that the valine at position 23 in NS4A was substituted with phenylalanine.

“Peptide” also includes fusion proteins, including, but not limited to, fusion proteins with a heterologous amino acid sequence, fusions with heterologous and homologous leader sequences, with or without N-terminal methionine residues; immunologically tagged proteins; and the like. Polypeptides can also be modified to, for example, facilitate attachment to a support (e.g., to a solid or semi-solid support, to a support for use as an array, and the liked).

The term “alkyl group” means a substituted or unsubstituted, saturated linear or branched hydrocarbon group or chain (e.g., C₁ to C₈) including, for example, methyl, ethyl, isopropyl, tert-butyl, heptyl, iso-propyl, n-octyl, dodecyl, octadecyl, amyl, 2-ethylhexyl, and the like. Suitable substituents include carboxy, protected carboxy, amino, protected amino, halo, hydroxy, protected hydroxy, nitro, cyano, monosubstituted amino, protected monosubstituted amino, disubstituted amino, C₁ to C₇ alkoxy, C₁ to C₇ acyl, C₁ to C₇ acyloxy, and the like.

The terms “active agent,” “inhibitor”, “drug” and “pharmacologically active agent” are used interchangeably herein to refer to a chemical material or compound which, when administered to an organism (human or animal) induces a desired pharmacologic and/or physiologic effect by local and/or systemic action. The pharmacologically active agents of interest in the present invention are viral protease inhibitors, particularly inhibitors of a viral NS3 protease, more particularly the HCV NS3 protease.

As used herein, the terms “treatment,” “treating,” and the like, refer to obtaining a desired pharmacologic and/or physiologic effect, such as reduction of viral titer(which may be the result of, for example, reduction of active NS3 in infected cells). The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease (e.g., reduction in viral titers).

The terms “individual,” “host,” “subject,” and “patient” are used interchangeably herein, and refer to a mammal, including, but not limited to, primates, including simians and humans, with humans being of particular interest, particularly those amenable to treatment according to the methods of the invention which involve administration of a viral protease inhibitor, particularly an HCV protease inhibitor, described herein (e.g., treatment of HCV infection using the inhibitors described herein).

### DETAILED DESCRIPTION OF THE INVENTION

Overview

The present invention is based on the discovery of inhibitors that decrease or render undetectable the activity of the HCV NS3 protease.

In general, the NS3 inhibitors of the invention are agents that decrease the efficiency or stability of the association of the NS3 and NS4A subunits of the HCV protease or interfere with substrate binding and or cleavage by NS3. Such inhibitors include agents that bind NS3 so as to decrease the efficiency or prevent NS4A binding; agents that bind NS4A so as to decrease the efficiency or prevent NS3 binding; and agents that are able to disrupt NS3-NS4A complexes. The NS3 protease inhibitors of present invention also include agents that bind NS3 so as to decrease the efficiency or prevent the binding and/or cleavage of NS3 substrates, and agents which interfere with NS3 substrate interactions.

In one embodiment, the inhibitors of the invention are derived from an optimized nine-amino acid minimal binding sequence of the domain of NS4A. (VVIVGRIVL), which correspond to residues 23-31 of native NS4A. Unlike previously described peptide inhibitors based on an amino acid sequence of NS4A, the inhibitors of the present invention lack the activation domain, and contain the minimal domain sufficient and necessary for binding to NS3 protease. “Bind” as used in the context of interaction of inhibitors described herein with an HCV viral protein (including polyprotein) is meant to refer to association of the inhibitor with the corresponding molecule so as to decrease its availability or its association with its native substrate (e.g., NS3 or NS4A). “Binding” can be mediated through any variety of interactions, including hydrophobic interaction, hydrophilic interaction, salt bridges, van der Waals associations, and the like.

In another embodiment, the inhibitors of the invention are derived from the “P” region of the amino acid sequence that serves as the NS3 cleavage substrate ((D/E)-(D/E)-X1-X2-X3-(C/S)). Specifically, the inhibitors have the amino acid sequence DEMEED and DEMEEE, or a derivative thereof (e.g., D-amino acids, acetylated residues, and the like), and comprise an acidic functionality at the P1 position. Of particular interest are the peptides Ac-DEMEED-OH and Ac-DEMEEE-OH. In general, these peptides are about 6 residues in length.

It is noted that the inhibitors find use not only in inhibition of HCV NS3 protease and thus treatment of HCV infection, but also in inhibition of viral proteases that share the relevant conserved NS3 protease domain or NS4A domains (e.g., the cleavage site, the NS3-binding domain of NS4A disclosed herein, or both). Such viruses include, but are not limited to, other flaviviruses such as dengue fever virus (see, e.g., Khumthong et al. J. Biochem Mol. Biol. 2002 March 35(2):206-12), and pestiviruses such as bovine viral diarrhea virus (see, e.g., Tautz et al. Virology Aug. 1, 2000; 273(2):351-63).

While the agents of the invention are referred to throughout the specification as HCV protease inhibitors, and particularly NS3 protease inhibitors, such reference is not meant to be limiting as to the mechanism of action, and applicants do not intend to be held to any particular theory as to such mechanism of action. For example, the agents of the invention may inhibit HCV replication by interaction with proteins other than NS3, particularly proteins that bind the same or similar target sequence from which the agents of the invention are designed. For example, several agents are described herein as inhibiting HCV replication by binding NS3 as a mimic of NS4A (e.g., mimicking binding of NS4A to NS3 in NS4A's role as an NS3 cofactor). However, other NS4A-mediated or -modulated activities can also be inhibited by such agents. For example, NS4A inhibits auto-cleavage of the NS2/NS3 polyprotein. Thus inhibitors that mimic NS4A binding may also have activity in inhibiting NS2/3 cleavage.

HCV Inhibitors

The inhibitors of the invention are generally peptides, where peptides include naturally-occurring and non-naturally occurring residues, . As noted above, peptides are defined herein as organic compounds comprising two or more amino acids covalently joined by an amide bond. Peptides may be referred to with respect to the number of constituent residues or amino acids or modified amino acids, i.e., a dipeptide contains two amino acid residues, a tripeptide contains three, etc. In general, the peptides described herein can be about 2, 4, 6, 7, 8, 9 or more residues in length, with peptides of from about 4 to about 6, from about 4 to about 7, and from about 4 to about 8 residues or about 5 to 9 residues being of particular interest. Peptides of less than 14 residues, less than 13 residues, less than 12 residues, less than 11 residues, or less than 10 residues are of particular interest. Where the peptides are bivalent, e.g., peptides attached via a linker peptide as described in more detail below, the bivalent peptide, excluding the residues of the linker peptide, is usually less than 20 residues, less than 19 residues, less than 18 residues, less than 17 residues, or less than 16 residues.

The peptides of the invention, while described herein as being composed of naturally occurring, L-amino acids, are not limited to such. The peptides described herein may be modified at the amino and/or carboxy termini; modified to contain the D-isomer rather than the normal L-isomer; modified chemically to have different substituents or additional moieties; and the like, with the proviso that these modifications do not eliminate or otherwise adversely affect the antiviral activity of the peptides. Exemplary chemical modifications of the peptides include acylation, alkylation, esterification, amidification, etc. to produce structural analogs.

In addition, the residues of the peptides described herein can be substituted with a non-encoded amino acid, or a modified amino acid. For example, cyclohexylalanine (“chA”, usually β-cyclohexylalanine), penacillamine (“Pen”), acetylated amino acid residues (“Ac”), amino acid residues modified by a tert group (“t”), hydroxyproline, nitrophenylalanine, homoarginine, thiazolidine, dehydroproline, aminobutyric acid (Aib), aminoisobutyric acid (Abu), and the like can be substituted for particular residues of a peptide.

Furthermore, the peptides described herein can be modified by amino acid insertion, deletion, addition, or substitution, again with the proviso that the modified peptide exhibits antiviral activity. The amino acid substitutions may be of a conserved or non-conserved nature. Conserved amino acid substitutions involve replacing one or more amino acids of the peptide sequences described herein with amino acids of similar charge, size, and/or hydrophobicity characteristics, such as, for example, a glutamic acid (E) to aspartic acid (D) amino acid substitution. Non-conserved substitutions involve replacing one or more amino acids with amino acids possessing dissimilar charge, size, and/or hydrophobicity characteristics, such as, for example, a glutamic acid (E) to valine (V) substitution.

Amino acid insertions may be insertions of single amino acid residues or stretches of residues. Insertions may be made at the carboxy or amino terminal end of the peptides (sometimes referred to as additions), as well as at a position internal to the peptide. Such insertions can range from 2 to 15 amino acids in length; in the present invention, shorter peptides (e.g., less than 10 amino acids) are preferred, as such are more likely to have access to the NS3 protease within infected cells.

Deletions of residues from the peptides described herein are also within the scope of the invention. Such deletions can involve removal of one or more amino acids from peptide sequence, with the lower limit length of the resulting peptide sequence being 2 or 3 to 4 amino acids. Such deletions may involve a single contiguous or greater than one discrete portion of the peptide sequences.

The peptides of the invention may be synthesized or prepared by techniques well known in the art. See, for example, Creighton, 1983, Proteins: Structures and Molecular Principles, W. H. Freeman and Co., N.Y., which is incorporated herein by reference in its entirety. Short peptides, for example, can be synthesized on a solid support or in solution. Longer peptides may be made using recombinant DNA techniques. Here, the nucleotide sequences encoding the peptides of the invention may be synthesized, and/or cloned, and expressed according to techniques well known to those of ordinary skill in the art. See, for example, Sambrook, et al., 1989, Molecular Cloning, A Laboratory Manual, Vols. 1-3, Cold Spring Harbor Press, N.Y.

The peptides of the invention may alternatively be synthesized such that one or more of the bonds which link the amino acid residues of the peptides are non-peptide bonds. These alternative non-peptide bonds may be formed by utilizing reactions well known to those in the art, and may include, but are not limited to imino, ester, hydrazide, semicarbazide, and azo bonds, to name but a few. In yet another embodiment of the invention, peptides comprising the sequences described above may be synthesized with additional chemical groups present at their amino and/or carboxy termini, such that, for example, the stability, bioavailability, and/or inhibitory activity of the peptides is enhanced. For example, hydrophobic groups such as carbobenzoxyl, dansyl, or t-butyloxycarbonyl groups, may be added to the peptides' amino termini. Likewise, an acetyl group or a 9-fluorenylmethoxy-carbonyl group may be placed at the peptides' amino termini. Additionally, the hydrophobic group, t-butyloxycarbonyl, or an amido group may be added to the peptides carboxy termini.

Further, the peptides of the invention may be synthesized such that their steric configuration is altered. For example, the D-isomer of one or more of the amino acid residues of the peptide may be used, rather than the usual L-isomer. Still further, at least one of the amino acid residues of the peptides of the invention may be substituted by one of the well known non-naturally occurring amino acid residues. Alterations such as these may serve to increase the stability, bioavailability and/or inhibitory action of the peptides of the invention. Any of the peptides described above may, additionally, have a non-peptide macromolecular carrier group covalently attached to their amino and/or carboxy termini. Such macromolecular carrier groups may include, for example, lipid-fatty acid conjugates, polyethylene glycol, or carbohydrates.

In general, the peptides of the subject invention are separated from their naturally occurring environment, and are preferably separated from compounds that would pose difficulties in compatibility with administration to a subject, particularly to a human subject. In certain embodiments, the peptide is present in a composition that is enriched for the protein as compared to a control.

Without being held to theory, as to the mode of action, exemplary peptide inhibitors are now described according to their proposed mechanism of NS3 inhibition.

Inhibitor Peptides which Interact with the Site on HCV NS3 which cleaves the NS4A/NS5B Junction.

In one embodiment, the HCV inhibitors are peptides that interact with the HCV NS3 protease so as to inhibit its ability to cleave the HCV polyprotein at the NS4A/NS5B cleavage site (substrate site), which cleavage normally results in production of NS4A. Thus, in one example, peptide inhibitors of this type can be based upon the polyprotein substrate of NS3, e.g., an amino acid sequence derived from the NS4A/NS5B cleavage site of the HCV polyprotein. The inhibitors of this type described herein were identified by screening combinatorial libraries based on the amino acid sequence of the NS4A/NS5B junction.

In one exemplary embodiment, peptide inhibitors of this class of NS3 protease inhibitors have the sequence derived from the six amino acids (P6-P1) on the N-terminus side of the NS4A/5B cleavage site and may have the following consensus sequence:

X1-DEMEE-X2

or

X1-DEMEEX2-X3

where, X1 represents an acetyl group or an integral number of residues from 0 to 7, generally from 0 to 5, usually from 0 to 4, preferably from 0 to 3, more preferably from 0 to 2, still more preferably 0 to 1, where X2 is an acidic amino acid (e.g. ASP, GLU) or a residue other than csyteine, and X3 represents an integral number of residues from 0 to 7, generally from 0 to 5, usually from 0 to 4, preferably from 0 to 3, more preferably from 0 to 2 or 0 to 1 amino acid residues; with the proviso that the peptide is of a suitable length such that the peptide inhibits NS3 activity (e.g., through binding to NS3) without activation of NS3. In one embodiment, the total number of residues provided by X1 and X2 (or by X1, X2, and X3) is less than 13, usually less than 12, more usually less then 11 or less than 10. Peptides comprising this motif and having about 5 to 9 residues, about 4 to 7 residues, or about 6 residues.

Exemplary peptides are DEMEED and DEMEEE, which possess an acidic functionality at the P1 position and are strong inhibitors of the NS3 protease. The compositions of the present invention comprise the peptides Ac-DEMEED-OH and Ac-DEMEEE-OH and derivatives thereof, such as esters, acidic functionality (e.g. difluoro-Leu) or other modifications which improve protease inhibition and/or delivery of these compositions in vivo.

Inhibitors that Interfere with NS3-NS4A Interaction

In other embodiments, the HCV NS3 protease inhibitors interfere with the binding and/or interaction of NS4A with NS3. This class of inhibitors preferably bind NS3 while exhibiting reduced NS3-activation activity relative to a wildtype NS4A peptide. Preferably, the inhibitor binds NS3 without eliciting significant or detectable activation of NS3 protease activity. NS3 protease inhibitors of this type described herein were identified by, for example, combinatorial libraries based on the site of NS4A which binds NS3, in particular the NS4A Val 23 or Val 26 residue.

In one exemplary embodiment, inhibitors of this class have the motif “GRI” with the following consensus sequence:

X1-GRI-X2

where, X1 represents an integral number of residues from 0 to 9, usually from 0 to 8, preferably from 0 to 6, more preferably from 0 to 4 residues, or 0 to 2 residues and

X2 represents an integral number of residues from 0 to 10, generally from 0 to 15, usually from 0 to 9, preferably from 0 to 6, more preferably from 0 to 4, or 0 to 2 residues.

with the proviso that the peptide is of a suitable length such that the peptide binds NS3 while exhibiting reduced NS3-activation activity relative to a wildtype NS4A peptide. Preferably, the inhibitor binds NS3 without eliciting significant or detectable activation of NS3 protease activity.

Peptides having this motif are of a total length of from about 4 to about 6, from about 4 to about 7, and from about 4 to about 8 residues, or about 5 to 9 residues are of particular interest. Peptides of less than 14 residues, less than 13 residues, less than 12 residues, less than 11 residues, or less than 10 residues are also of particular interest. . In one embodiment the total residues of X1 and X2 is less than 9 residues.

Specific exemplary peptides having this X1-GRI-X2 consensus sequence include, but are not necessarily limited to: VVIVGRI, VIVGRIVL, IVGRIVL, VGRIVL, GRIVL. Any residues of these peptides may be either an L- or a D-isomer, with G substituted by a D-amino acid (e.g., VVIVxRI, VIVxRIVL, IVxRIVL, VxRIVL, xRIVL, where lowercase “x” denotes a D-amino acid residue).

In further embodiments, X1 is LVIV, t-LVIV, CVIV, PenVIV, or FVIV, with FVIV being preferred. In still other embodiments, X1 is C, Q,E, F, ChA, W, R, or Y, or a D isomer thereof (e.g., c, q, e, f, chA, w, r, or y). In one embodiment, X2 is an carboxyl group, or V, VL, or a D-amino acid thereof. Peptides of the invention may optionally comprise one or more, usually one, two, or three, lysine residues (or other charged residue) at X1, X2, or both X1, and X2 to provide for improved solubility.

In still another exemplary embodiment, the inhibitors have the formula:

X1-Z1-Z2-Z3-Z4-X2

where X1 is an integral number of residues from 0 to 5, Z1 is a hydrophobic residue, preferably a large hydrophobic residue, Z2 is a D-isomer of a hydrophobic residue, preferably a D-isomer of a large hydrophobic residue, Z3 is a positively charged residue, preferably a large positively charged residue; Z4 is a hydrophobic residue or a positively charged residue; and X2 is an integral number of residues from 0 to 3, wherein X2 may optionally comprises a terminal amino group. In specific embodiments, Z1 is M, F, W, or Cha, preferably Cha; Z2 is a D-amino acid of v, a, l, i, p, m, f, w, or chA; Z3 is K, R, or H, preferably K or R; and Z4 is any of Z1 or Z3.

Where X1 has two or more residues, the residue position two residues N-terminal to the residue at Z1 is preferably not a negatively charged residue (e.g., D or E) or an amide (e.g., N or Q). Z1 is preferably not a negatively charged residue (e.g., D or E) or an amide (e.g., N or Q). Furthermore, Z2 and Z3 are preferably not a negatively charged residue (e.g., D or E). Further exemplary peptides have the motif:

X1-Cha-v-(R/K)-(Cha/R)-X2

where X1 is an integral number of residues from 0 to 5, generally from 0 to 3, usually from 0 to 2 or 0 to 1, preferably from 0 to 3, more preferably from 0 to 1, with W, R, FFR, FNW, FNR, or FFW being exemplary; X2 is an integral number of residues from 0 to 3, more preferably from 0 to 1, with an amino group or I-Cha being preferred; “tv” represent D-Valine, and Cha is α- or β-cyclohexylalanine, with β-cyclohexyalanine being preferred, where Cha may be either in the L- (“Cha”) or D-isomer (“chA”) form. X2 may optionally comprise a terminal amino group (NH2) (e.g., to improve solubility).

Peptides having the X1-Z1-Z2-Z3-Z4-X2 motif or, more specifically, the X1-Cha-v-(R/WK)-(Cha/R)-X2 motif are of a length of from about 4 to about 6, from about 4 to about 7, and from about 4 to about 8 residues, or about 5 to 9 residues are of particular interest. Peptides of less than 13 residues, less than 12 residues, less than 11 residues, or less than 10 residues, less than 9 residues, or less than 8 residues are also of particular interest. In one embodiment the total residues of X1 and X2 is less than 9 residues. Exemplary peptides of this motif include W-Cha-v-R-Cha-I-Cha; R-Cha-v-R-Cha-I-Cha; F-F-W-Cha-v-R; W(Cha)vR(Cha)I(Cha)-NH2; R(Cha)vR(Cha)I(Cha)-NH2; and FFW(Cha)vR(Cha)-NH2. Peptides of the invention may optionally comprise one or more, usually one, two, or three, lysine residues (or other charged residue) at X1, X2, or both X1, and X2 to provide for improved solubility.

Modification of Peptide Inhibitors to Enhance Cellular Uptake

In one embodiment, the peptides described herein are associated with a molecule that enhances cellular uptake and/or provides for enhanced activity in inhibition of NS3 protease. In an embodiment of particular interest, at least two peptides are linked by a third amino acid sequence. For example, such linked peptides can have the general formula:

Peptide1-L-Peptide2

where Peptide 1 can be selected from any of the peptides described herein,

Peptide 2 can be selected from any of the peptides described herein, and

L represents a linker, such as an amino acid sequence, that links Peptides 1 and 2.

Where L is an amino acid sequence, L is generally primarily composed of polar amino acid residues, preferably cationic polar residues, e.g., arginine, lysine, and asparagine. The linker may be of any suitable length, generally from at least 2 residues to about 9 residues, and may be as long as 15 residues, with the proviso that the total length of molecule represented by Peptide1-L-Peptide2 is not such that cellular uptake is compromised. In certain embodiments, the linker comprises a D-arginine oligomer, and in particular, a D-arginine oligomer of about 4, 5, 6, 7, 8, or 9 arginine residues, with 9 being of particular interest. Generally L provides for covalent linking of Peptide 1 to Peptide 2.

Peptide 1 and Peptide 2 may be the same or different in amino acid sequence, and may be designed to bind to the same or different targets on NS3 (e.g., to bind to the site of NS3 responsible for cleavage of the polyprotein or to the site on NS3 that interacts with NS4A). In a preferred embodiment, Peptide1 and Peptide2 are designed to bind to different targets on NS3.

In certain embodiments, compositions of the present invention comprise a linker of nine arginine residues which connects the two types of NS3 protease inhibitors together which allows the inhibition of NS3 to be achieved on two fronts, one interfering with the NS3 protease cleavage site and the other inhibitor interfering with NS4A binding. The linker molecule allows for an increase NS3 inhibitory effect and greater efficacy.

The polypeptides of the present invention may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

Screening the HCV Protease Inhibitors for Protease Inhibitor Activity

The protease inhibitors of the present invention can be screened for HCV antiviral activity using any of a variety of in vitro assays known in the art. In addition, the inhibitors of the present invention can be screened for HCV antiviral activity in vivo, using the animal model of HCV infection described in WO 20/0167854.

Antibodies that Specifically Bind Peptide Inhibitors

Antibodies that specifically bind to a peptide described herein are also of interest. As used herein, the term “antibody” refers to a polypeptide or group of polypeptides which are comprised of at least one antibody combining site. An “antibody combining site” or “binding domain” is formed from the folding of variable domains of an antibody molecule(s) to form three-dimensional binding spaces with an internal surface shape and charge distribution complementary to the features of an epitope of an antigen, which allows an immunological reaction with the antigen. An antibody combining site may be formed from a heavy and/or a light chain domain (V_{H} and V_{L}, respectively), which form hypervariable loops which contribute to antigen binding. The term “antibody” includes, for example, vertebrate antibodies, hybrid antibodies, chimeric antibodies, altered antibodies, univalent antibodies, the Fab proteins, and single domain antibodies.

Methods for production of antibodies that specifically bind a selected antigen are well known in the art. Immunogens for raising antibodies can be prepared by mixing an peptide of interest with an adjuvant, and/or by making fusion proteins with larger immunogenic proteins. Peptides can also be covalently linked to other larger immunogenic proteins, such as keyhole limpet hemocyanin. Immunogens are typically administered intradermally, subcutaneously, or intramuscularly to experimental animals such as rabbits, sheep, and mice, to generate antibodies. Monoclonal antibodies can be generated by isolating spleen cells and fusing myeloma cells to form hybridomas.

Preparations of polyclonal and monoclonal antibodies specific for polypeptides encoded by a selected polynucleotide are made using standard methods known in the art. Typically, at least 5, 8, 10, or 12 contiguous residues are required to form an epitope. Epitopes that involve non-contiguous residues may require a longer polypeptide, e.g., at least 15, 25, or 50 residues. Antibodies that specifically bind to peptides of the invention are generally those that provide a detection signal at least 5-, 10-, or 20-fold higher than a detection signal provided with a peptide of different sequence when used in Western blots or other immunochemical assays. Preferably, antibodies that specifically bind peptides of the invention do not bind to other proteins in immunochemical assays at detectable levels and can immunoprecipitate the specific polypeptide from solution.

As noted above, “antibodies” encompasses various kinds of antibodies, including, but not necessarily limited to, naturally occurring antibodies, single domain antibodies, hybrid antibodies, chimeric antibodies, single-chain antibodies, and antibody fragments that retain antigen binding specificity, and the like. The invention also encompasses single domain antibodies, hybrid antibodies, chimeric antibodies, single-chain antibodies, and antibody fragments that retain antigen binding specificity. As used herein, a “single domain antibody” (dAb) is an antibody which is comprised of an VH domain, which reacts immunologically with a designated antigen. A dAb does not contain a V_{L} domain, but may contain other antigen binding domains known to exist in antibodies, for example, the kappa and lambda domains. Methods for preparing dAbs are known in the art. See, for example, Ward et al. (1989). Antibodies may also be comprised of V_{H} and V_{L} domains, as well as other known antigen binding domains. Examples of these types of antibodies and methods for their preparation are known in the art (see, e.g., U.S. Pat. No. 4,816,467, which is incorporated herein by reference), and include the following.

Included also within the definition of antibodies are “Fab” fragments of antibodies. The “Fab” region refers to those portions of the heavy and light chains which are roughly equivalent, or analogous, to the sequences which comprise the branch portion of the heavy and light chains, and which have been shown to exhibit immunological binding to a specified antigen, but which lack the effector Fc portion. “Fab” includes aggregates of one heavy and one light chain (commonly known as Fab′), as well as tetramers containing the 2H and 2L chains (referred to as F(ab).sub.2), which are capable of selectively reacting with a designated antigen or antigen family. “Fab” antibodies may be divided into subsets analogous to those described above, i.e, “vertebrate Fab”, “hybrid Fab”, “chimeric Fab”, and “altered Fab”. Methods of producing “Fab” fragments of antibodies are known within the art and include, for example, proteolysis, and synthesis by recombinant techniques.

Pharmaceutical Compositions

The above-discussed compositions can be formulated using well-known reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, a HCV protease inhibitor is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, the HCV protease inhibitors and ribavirin are generally administered to individuals in a formulation (e.g., in the same or in separate formulations) with a pharmaceutically acceptable excipient(s). The therapeutic HCV protease inhibitor agents and ribavirin, as well as additional therapeutic agents as described herein for combination therapies, can be administered orally, subcutaneously, intramuscularly, parenterally, or other route. HCV protease inhibitor agent and ribavirin may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

The therapeutic agent(s) may administered in a unit dosage form and may be prepared by any methods well known in the art. Such methods include combining the compounds of the present invention with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant invention can be used. A wide variety of delivery devices and systems are known to those skilled in the art.

Although such may not be necessary, HCV protease inhibitor agents described herein can optionally be targeted to the liver, using any known targeting means. The protease inhibitors of the invention may be formulated with a wide variety of compounds that have been demonstrated to target compounds to hepatocytes. Such liver targeting compounds include, but are not limited to, asialoglycopeptides; basic polyamino acids conjugated with galactose or lactose residues; galactosylated albumin; asialoglycoprotein-poly-L-lysine) conjugates; lactosaminated albumin; lactosylated albumin-poly-L-lysine conjugates; galactosylated poly-L-lysine; galactose-PEG-poly-L-lysine conjugates; lactose-PEG-poly-L-lysine conjugates; asialofetuin; and lactosylated albumin.

The terms “targeting to the liver” and “hepatocyte targeted” refer to targeting of an NS3 inhibitor agent to a hepatocyte, particularly a virally infected hepatocyte, such that at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, of the protease inhibitor agent administered to the subject enters the liver via the hepatic portal and becomes associated with (e.g., is taken up by) a hepatocyte.

HCV infection is associated with liver fibrosis and in certain embodiments the protease inhibitors may by useful in treating liver fibrosis (particularly preventing, slowing of progression, etc.). The methods involve administering a protease inhibitor of the invention as described above, in an amount effective to reduce viral load, thereby treating liver fibrosis in the subject. Treating liver fibrosis includes reducing the risk that liver fibrosis will occur; reducing a symptom associated with liver fibrosis; and increasing liver function.

Whether treatment with protease inhibitor agent as described herein is effective in reducing liver fibrosis is detenmined by any of a number of well-established techniques for measuring liver fibrosis and liver function. The benefit of anti-fibrotic therapy can be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

Treatment of liver fibrosis (e.g., reduction of liver fibrosis) can also be determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

In some embodiments, a therapeutically effective amount of a protease inhibitor agent of the invention is an amount of protease inhibitor agent that effects a change of one unit or more in the fibrosis stage based on pre- and post-therapy measures of liver function (e.g, as determined by biopsies). In particular embodiments, a therapeutically effective amount of protease inhibitor reduces liver fibrosis by at least one unit in the Child-Pugh, METAVIR, the Knodell, the Scheuer, the Ludwig, or the Ishak scoring system.

Secondary, or indirect, indices of liver function can also be used to evaluate the efficacy of treatment. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score. An effective amount of protease inhibitor agent is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of protease inhibitor agent is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Qualitative or quantitative tests of functional liver reserve can also be used to assess the efficacy of treatment with protease inhibitor agent. These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of protease inhibitor agent in this context can be regarded as an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with protease inhibitor agent is effective in reducing the incidence of a disorder associated with cirrhosis of the liver can readily be determined by those skilled in the art.

Reduction in HCV viral load, as well as reduction in liver fibrosis, can be associated with an increase in liver function. Thus, the invention provides methods for increasing liver function, generally involving administering a therapeutically effective amount of a protease inhibitor of the invention. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, λ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal range of alanine transaminase is from about 7 to about 56 units per liter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of protease inhibitor agent in this context is one that is effective to increase liver function by at least about 10%, at least about 20%, at least about 30%, at least about 40%, at least about 50%, at least about 60%, at least about 70%, at least about 80%, or more. For example, a therapeutically effective amount of protease inhibitor agent is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of protease inhibitor is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

HCV infection is associated with hepatic cancer and in certain embodiments, the present invention provides compositions and methods of reducing the risk that an individual will develop hepatic cancer. The methods involve administering an protease inhibitor agent, as describe above, wherein viral load is reduced in the individual, and wherein the risk that the individual will develop hepatic cancer is reduced. An effective amount of protease inhibitor agent is one that reduces the risk of hepatic cancer by at least about 10%, at least about 20%, at least about 25%, at least about 30%, at least about 35%, at least about 40%, at least about 45%, at least about 50%, at least about 55%, at least about 60%, at least about 65%, at least about 70%, or more. Whether the risk of hepatic cancer is reduced can be determined in, e.g., study groups, where individuals treated according to the methods of the invention have reduced incidence of hepatic cancer.

Subjects Amenable to Treatment Using the Agents of the Invention

Individuals who have been clinically diagnosed as infected with a hepatitis virus, particularly HCV, are suitable for treatment with the methods of the present invention. Individuals who are infected with HCV are generally identified (diagnosed) as having HCV RNA in their blood, and/or having anti-HCV antibody in their serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1, or other HCV subtypes and quasispecies. Such individuals include naïve individuals (e.g., individuals not previously treated for HCV) and individuals who have failed prior treatment for HCV (“treatment failure” patients). Treatment failure patients include non-responders (e.g., individuals in whom the HCV titer was not significantly or sufficiently reduced by a previous antiviral treatment for HCV); and relapsers (e.g., individuals who were previously treated for HCV, whose HCV titer decreased, and subsequently increased). In particular embodiments of interest, individuals of interest for treatment according to the invention have an HCV titer of at least about 10⁵, at least about 5×10⁵, or at least about 10⁶, or greater than 2 million genome copies of HCV per milliliter of serum.

Determining Effectiveness of Antiviral Treatment

Whether a subject method is effective in treating a hepatitis virus infection, particularly an HCV infection, can be determined by measuring viral load, or by measuring a parameter associated with HCV infection, including, but not limited to, liver fibrosis.

Viral load can be measured by measuring the titer or level of virus in serum. These methods include, but are not limited to, a quantitative polymerase chain reaction (PCR) and a branched DNA (bDNA) test. For example, quantitative assays for measuring the viral load (titer) of HCV RNA have been developed. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329.

As noted above, whether a subject method is effective in treating a hepatitis virus infection, e.g., an HCV infection, can be determined by measuring a parameter associated with hepatitis virus infection, such as liver fibrosis. Liver fibrosis reduction can be assessed by a variety of serum-based assay or by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems. Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

As one non-limiting example, levels of serum alanine aminotransferase (ALT) are measured, using standard assays. In general, an ALT level of less than about 45 international units per milliliter serum is considered normal. In some embodiments, an effective amount of protease inhibitor is an amount effective to reduce ALT levels to less than about 45 IU/ml serum.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Example 1

HCV NS3 Protease Inhibitors Based in the NS4A/N5B Cleavage Junction Site.

An assay using frontal affinity chromatography (FAC) with an in-line mass spectrometer (MS) as described by Schriemer et al. 1998 Agnew. Chem. Int. Ed. 37, 3383-3387 (referred to as FAC-MS) was adapted to identify NS3 protease inhibitors. Briefly, a HCV NS3 protease domain was cloned into a site in a vector that allows for the recombinant NS3 protein to be biotinylated when expressed in E. coli. This provides for immobilization of the NS3 protease domain on streptavidin coated beads which are packed into a miniaturized column. The column is connected in-line to a mass spectrometer. This arrangement facilitates assaying combinatorial compounds individually from a library of peptides for activity in binding the immobilized NS3 protease domain. As the library is continually infused through the column, each compound produces a sigmoidal curve as its elution profile.

To determine the validity of the FAC-MS assay, a model combinatorial library was made utilizing the peptide sequence of the NS4A/5B junction which is a site of cleavage by NS3 protease:

(D/E)-(D/E)-X₁-X₂-X₃-(C/S)Δ(A/T)-X₄-X₅-X₆

As discussed above, the convention for any protein cleavage site of the substrate may be represented as:

P₆-P₅-P4-P3-P₂-P₁-Δ-P₁′-P₂′-P₃′-P₄′

The combinatorial library was produced by randomizing the P₁ position of the P₆-P₁ NS4A/5B cleavage junction sequence:

P₆-P₁ sequence of NS4A/5B junction; D-E-M-E-E-C-OH

Library randomized in P₁ position: Ac-D-E-M-E-E-X-OH

where X was randomized to: Gly; Ser, Phe, D-Cys, L-Cys, Asp, Lys, Ala; and Ac refers to an acetyl group.

This model library represented a broad range of chemical functionalities. The controls were the D/L-Cys in position P1. Although these residues have the same molecular mass, the NS3 enzyme does not recognize the D-Cys peptide as well as the L-Cys containing peptide.

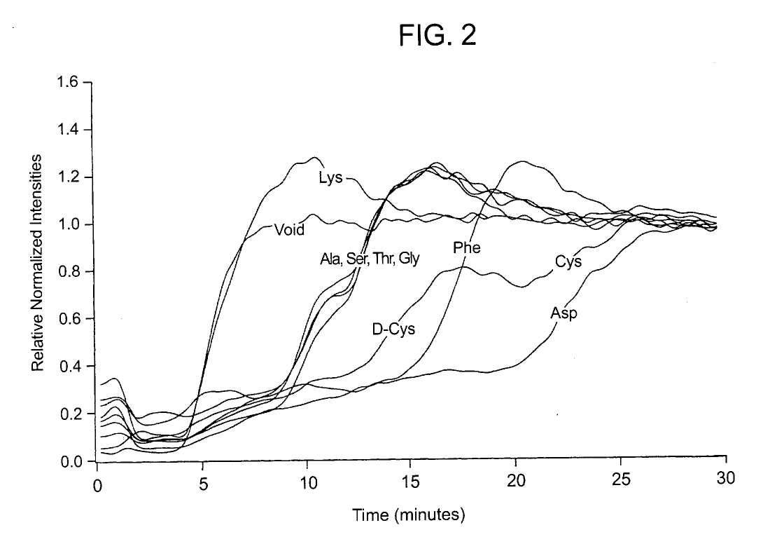

These two molecules, peptides with D-Cys (“D-Cys”) or L-Cys (“L-Cys”) at P₁, were resolved by FAC-MS, giving the characteristic “step” function in their elution profile. One unique result was that the Asp-(Ac-DEMEED-OH) peptide (“Asp”) also eluted very late as shown in the FAC-MS elution profile of FIG. 2. The order of elution was as follows: void volume marker, K, G, S, A, D-Cys, F, D, L-Cys. This late elution profile was indicative of NS3 protease inhibitor activity. This is one of the first NS3 protease inhibitors discovered which comprises an acidic functional group in the critical P₁ position.

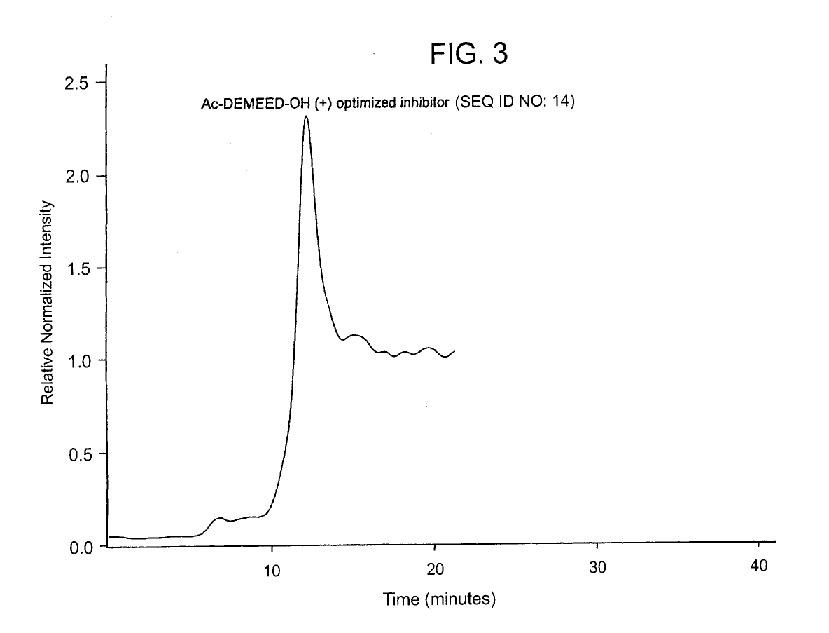

To further substantiate that the Ac-DEMEED-OH peptide was binding or interfering with NS3, a competitive assay was performed with a known protease inhibitor. The known protease inhibitor was added to the library and the elution time of the Ac-DEMEED-OH peptide was monitored, all other conditions being the same as the assay for Ac-DEMEED-OH alone. The elution time shifted forward, thereby substantiating the conclusion that the Ac-DEMEED-OH peptide was a novel substrate based inhibitor to the HCV NS3 protease (FIG. 3).

This Ac-DEMEED-OH molecule was verified as a genuine NS3 protease inhibitor by solution phase enzyme kinetics. Lineweaver-Burke analysis yielded the following inhibitor constants in the absence of the activating cofactor peptide from NS4A.

TABLE 1
Inhibitor Constants for NS3 Protease Inhibitors

| Peptide: | Kₗ (mM): |
|---|---|
| Ac-DEMEEC-OH | 0.454 |
| Ac-DEMEED-OH | 1.67 |
| Ac-DEMEEE-OH | 1.52 |

In similar experiments to those completed for the Ac-DEMEED-OH molecule, the Ac-DEMEEE-OH, which likewise has an acidic function in the P₁ position, was also identified as a NS3 protease inhibitor.

In summary, the peptides Ac-DEMEED-OH and Ac-DEMEEE-OH, which possess an acidic functionality at the P₁ position are novel inhibitors of the NS3 protease that have an apparently unique mechanism of action. The compositions of the present invention comprise the peptides Ac-DEMEED-OH and Ac-DEMEEE-OH and any derivatives of these peptides, such as esters or other modifications which improve delivery of these compositions in vivo as well as mimetics of the acidic functionality (e.g. difluoro-Leu).

### Example 2

Identification of a NS4A Mutants that Retain NS3 Binding

The first cleavage that NS3 performs liberates its co-factor peptide NS4A, a 54 residue peptide that binds and activates NS3. A 12 residue peptide corresponding to a conserved region of NS4A has been shown to be sufficient for binding and activation of the protease domain (CVVIVGRIVLSG). The present inventors undertook to identify peptides that bind NS3 more tightly than wild-type (wt) NS4A, and at the same time activated the cleavage of substrate by NS3 to a lesser degree than wt NS4A.

The inventors first examined the ability of mutant NS4A peptides to bind to NS3 using the FAC-MS assay described in Example 1. The sequence of the peptides described here is described by the wild-type residue modified, the position of the residue in the wildtype NS4A polypeptide, and the residue substituted at that position. Thus, for example V23F indicates that the valine at position 23 in NS4A was substituted with phenylalanine. “Pen” refers to Penacillamine; “chA” refers to β-cyclohexylalanine; “t” refers to “tert” (so that, for example, “t-L” refers to tert-leucine.

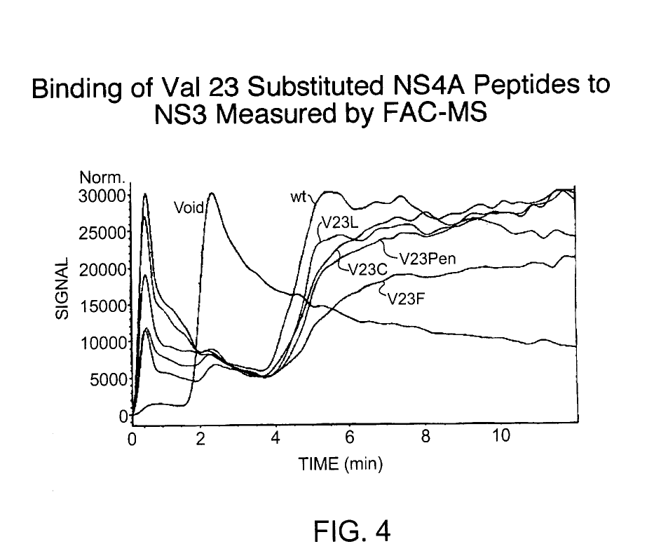

In the context of full length binding and activation domain of NS4A, two different sites were mutated in NS4A: Valine 23_{NS4A} and Valine 26_{NS4A}. In analysis of the role of Valine23 of NS4A, a series of peptides with amino acid substitutions in position 23 (the first valine in the KKGSVVIVGRIVLSGK sequence, where lysines are added at the N- and C-terminus relativ ot the native sequence to improve solubility) were synthesized, equimolar amounts of the peptides mixed, and the mixture subjected to FAC-MS analysis. The results for the NS4A peptide V23L, V23C, V23Pen, and V23F are provided in FIG. 4. As noted earlier, the later the elution time, the more tightly bound the peptide to the NS3 on the column. Of the peptides in FIG. 4, , the V23F_{NS4A} peptide eluted last (bound the most tightly to NS3).

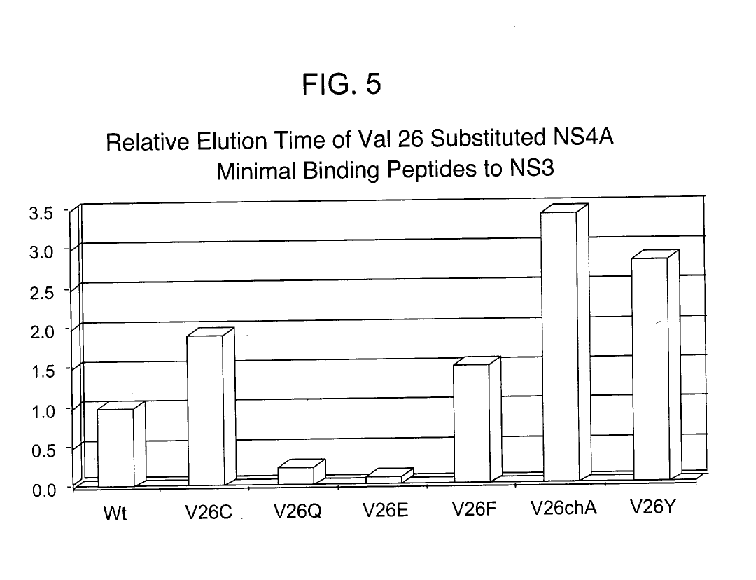

In analysis of the role of Valine 26 of NS4A, peptides of VVIXGRIVL (where X=V (wildtype), C, Q, E, F, chA, or Y) were synthesized and subjected to FAC-MS analysis as described above. The results of FAC-MS analysis of the V26NS4A mutant peptides is shown in FIG. 5. Relative binding affinities were calculated by measuring the difference between the void volume and the elution time of the peptide.

In short, the larger the side chain of the residue in the V23_{NS4A} mutant peptide, the more tightly the peptide bound NS3, with the exception of Cys. Despite the steric clashes predicted by modeling, the large hydrophobic residues in the NS4A peptides bound to NS3 most tightly. This indicates that there is a significant amount of flexibiltiy in both the Val23 and Val26 pockets.

Binding of the peptides was then analyzed by enzyme kinetics to detect whether the mutated NS4A peptides activated NS3-catalysed cleavage of substrates individually. Two NS3 substrates were used: 5ApNA (which contains the P side residues (six of them) of the substrate linked to para-nitroaniline) and 5A/5B (which is the natural cleavage junction that is found between the NS5A and NS5B proteins) using conventional methods (see, e.g., Landro et. al. 1997, Biochemistry 36 p9340-9348; and “Enzyme Kinetics” by Irwin Segel pub: Wiley and Sons, New York 1975 Chapter 5). The results are summarized in Tables 2 and 3 below. Values in the tables were obtained by extrapolating the Kₘ and k_{cat} values obtained for substrates at several concentrations of NS4A mutant peptides to infinite NS4A concentration. The Kₘ and k_{cat} values were obtained from double reciprocal plots of initial velocity measurements made at several concentrations of substrate and a constant NS4Aconcentration. Peptide concentrations used in calculations in Table 3 are based on those supplied by the facility that synthesized the peptides at the inventors' direction. Peptides concentrations for values in Table 2 were independently confirmed. Two different batches of NS3 that had different specific activities were used with the 5A/5B substrate in Table 3, one for the V23 mutant peptides and one for the V26 mutant peptides.

TABLE 2
Activation of NS3 by Mutant NS4A
peptides using 5A-pNA as the substrate

| | | | k_{cat}/Kₘ |
|---|---|---|---|
| NS4A peptide | Kₘ (mM) | k_{cat} (min⁻¹) | (mM⁻¹ min⁻¹) (^{a}) |
| None | 1.3 ± 0.3 | 0.31 ± 0.04 | 0.237 |
| Wild-type | 0.172 ± 0.001 | 1.02 ± 0.1 | 5.91 (25) |
| V23C | 0.25 ± .03 | 1.18 ± 0.01 | 4.72 (20) |
| V23L | 0.0812 ± .0009 | 0.94 ± 0.045 | 11.6 (49) |
| V23tert-L | 0.228 ± .004 | 0.73 ± 0.03 | 3.20 (14) |
| V23Pen | 0.27 ± .01 | 0.87 ± 0.09 | 3.25 (14) |
| V23F | 0.62 ± .04 | 0.469 ± 0.03 | 0.472 (3.1) |
| V26C | 0.16 ± .01 | 0.90 ± 0.05 | 5.74 (24) |
| V26Q | 0.347 ± .005 | 0.40 ± 0.07 | 1.15 (4.8) |
| V26F | 0.62 ± 0.04 | 0.47 ± 0.03 | 0.742 (3.1) |
| V26Y | 0.78 ± 0.04 | 0.54 ± 0.04 | 0.694 (2.9) |
| V26W | 1.20 ± 0.07 | 1.00 ± 0.04 | 0.834 (3.5) |
| V26Cha | 1.0 ± 0.1 | 1.2 ± 0.1 | 1.18 (4.9) |
| V26R | 1.51 ± 0.05 | 0.97 ± 0.04 | 0.641 (2.7) |

TABLE 3
Activation of NS3 by Mutant NS4A
peptides Using 5A/5B as the substrate

| | | | k_{cat}/Kₘ |
|---|---|---|---|
| NS4A peptide | Kₘ (mM) | k_{cat} (min⁻¹) | (mM⁻¹ min⁻¹) (^{a}) |
| None | 0.10 ± 0.01 | 1.86 ± 0.09 | 18.8 |
| Wild-type | 0.024 ± .003 | 4.6 ± 0.2 | 190 (10) |
| V23C | 0.024 ± .002 | 7.45 ± 0.02 | 307 (16) |
| V23Pen | 0.026 ± 0.003 | 6.8 ± 0.6 | 261 (14) |
| V23F | 0.086 ± 0.005 | 5.3 ± 0.3 | 61.9 (3.3) |
| None | 0.067 ± 0.004 | 9.4 ± 0.2 | 140 |
| V26C | 0.056 ± 0.01 | 22 ± 3 | 410 (2.9) |
| V26F | 0.12 ± 0.02 | 13 ± 5 | 116 (0.83) |
| V26W | 0.14 ± 0.01 | 16.0 ± 0.9 | 111 (0.79) |
| V26Cha | 0.37 | 34.1 | 906 (6.5) |
| V26R | 0.19 ± 0.01 | 20 ± 2 | 103 (0.74) |

^{a} The increase in the value of k_{cat}/Kₘ over that of NS3 alone is shown in brackets.

At position 23, the V23F_{NS4A} peptide was the ‘poorest’ activator of NS3 catalysed cleavage, (Table 2). The lack of activation was due to both a near absence of increase in k_{cat} as well as a smaller decrease in the value of Kₘ of NS3 for substrates.

When the same kinetic analysis was performed with the mutant NS4A peptides with mutations at position 26, it was discovered that large residues at this position resulted in an increase in the value of Kₘ of NS3 for 5A-pNA over that observed in the absence of added NS4A. When the substrate 5A-pNA was used the increase in Kₘ was accompanied by an increase in the value of k_{cat} over what was seen in the presence of wt NS4A. However when the same measurements were performed using the natural NS3 substrate, the value of kcat of NS3 for 5A/B did not rise above what was seen with wt NS4A. This indicated that the correct conformation of the natural substrate, when bound to NS3, is influenced by the residue at position 26 in NS4A. In addition, these data indicate that the proper substrate must be used when screening for an effect of mutated NS4A peptides on NS3 cleavage. Thus use of the colorometric substrate 5A pNA in such screening assays may not successfully detect NS3 inhibitors of interest. At position 26, the V26R_{NS4A} peptide was the poorest activator of NS3 catalysed cleavage. The lack of activation in this case was mostly due to an increase in the value of the Kₘ of NS3 for substrates.

Exemplary peptides were analyzed to compare how tightly bound the peptides are to NS3. The results, provided in Table 4 below, indicate that results obtained from the FAC-MS correlate well with the values obtained from kinetic analysis.

TABLE 4
Summary of the Kₐ and K_{d} values of NS3 for NS4

| Peptide | Ka (μM) | Kd (μM) |
|---|---|---|
| WT NS4A | 5.7 ± 0.6 | 6.3 |
| V23L | 2.71 ± 0.08 | 5.1 |
| V23t-L | 1.7 ± 0.2 | ND |
| V23Pen | 3.7 ± 0.8 | 4.7 |
| V23C | 11 ± 4 | 4.8 |
| V23F | 1.1 ± 0.3 | 3.7 |

Table 4 shows the Kₐ (μM) and K_{d} (μM) values of NS3 binding to wildtype NS4A and the NS4A peptides. The Kₐ values were obtained from analysis of kinetic data. The K_{d} values were obtained using FAC-MS from FIG. 4. (Schriemer, D C et al 1998 Agnew. Chem Int ed 37, 3383-3387). Table 2 indicates that the binding constants for the mutated peptides are better than that of wild-type NS4A and thus further improvements can be made in order to achieve a peptide that can displace the NS4A from its site in vivo and thus inhibit the NS3 protease.

### Example 3

Identification of a Minimal Domain of NS4A that Binds NS3

The FAC-MS assay described above was used to determine the minimal domain in NS4A that is required to bind to NS3. Two libraries were generated: one where each consecutive N-terminal residue of VVIVGRIVLSGK was deleted; and one where each consecutive C-terminal residue of GCVVIVGRIVL was been deleted. The libraries were as follows:

| N-Terminal Deletion Library | C-Terminal Deletion Library |
|---|---|
| LSGK | GCVV |
| VLSGK | GCVVI |
| IVLSGK | GCVVIV |
| RIVLSGK | GCVVIVG |
| GRIVLSGK | GCVVIVGR |
| VGRIVLSGK | GCVVIVGRI |
| IVGRIVLSGK | GCVVIVGRIV |
| VIVGRIVLSGK | GCVVIVGRIVL |
| VVIVGRIVLSGK | |

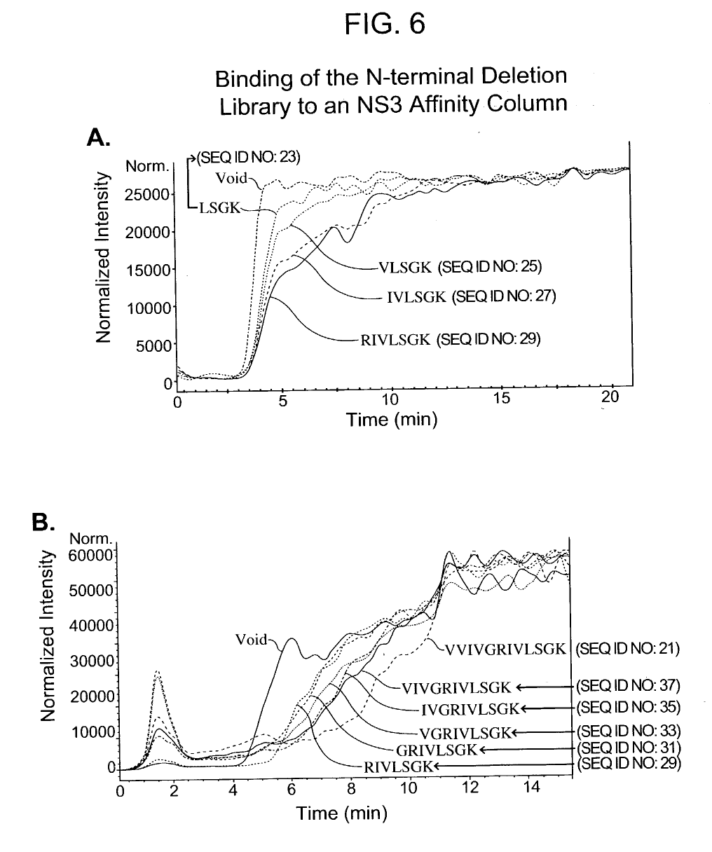

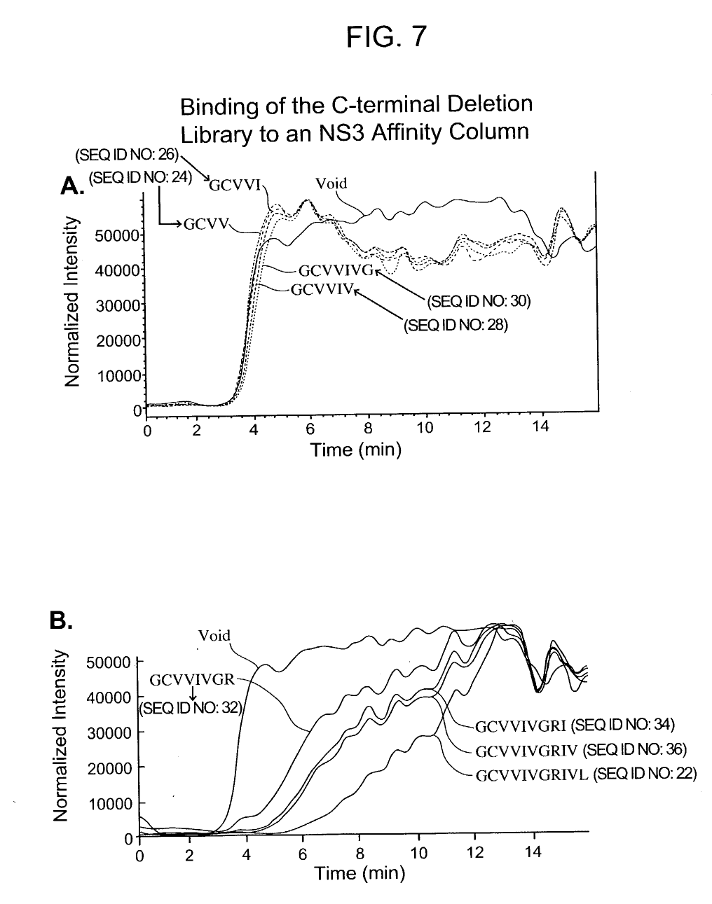

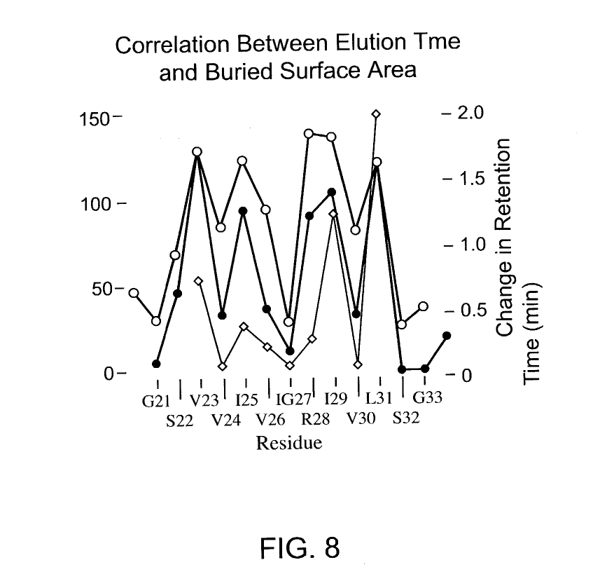

Each of the libraries was subjected to FAC-MS analysis The results for an exemplary run are shown in FIG. 6 (N-terminal deletion library) and FIG. 8 (C-terminal deletion library). In each of FIGS. 6 and 7, the elution profiles of peptides that did not bind well are shown Panel A, while the peptides having significant elution times are shown in Panel B. The buried surface area of each NS4A residue in the NS3/NS4A complex was compared with the change in elution time caused by each successive NS4A deletion peptide (FIG. 8). Analysis of the FAC-MS results indicated that there was a correlation between the amount of buried surface area of a residue (as indicated by the crystal structure) and the loss in retention time in FAC-MS (FIG. 8).

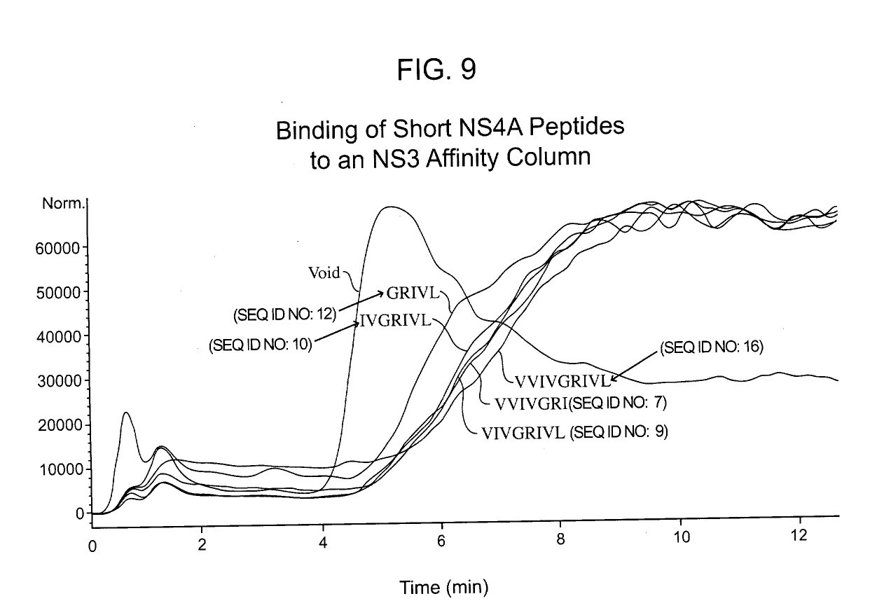

Using the information from the deletion libraries, several small peptides were made (VVIVGRI, VIVGRIVL, IVGRIVL, VGRIVL, and GRIVL) and again examined their binding to NS3 using FAC-MS. The results are show in FIG. 9. The binding of widltype NS4A in this system was measured and found to be 6.3 μM; the peptides gave values between 88 μM and 178 μM. (Table 4).

The ability of the short peptides to activate the NS3 protease was tested at several concentrations between 66 and 360 μM peptide. The concentration of substrate 5A-pNA was subsaturating. Table 5 shows the NS3 activity rates in the presence and absence of exemplary protease inhibitor peptides.

TABLE 5
Rates of NS3 in the presence of short NS4A peptides.

| | Rate | |
|---|---|---|
| Peptide | (μmoles/min/mg) | K_{d} (μM) |
| No NS4A | 3.68 | — |
| VVIVGRI | 1.88 | 103 |
| VIVGRIVL | 1.76 | 88 |
| IVGRIVL | 1.80 | 103 |
| VGRIVL | 1.70 | — |
| GRIVL | 1.76 | 178 |
| Wild Type NS4A | 37.4 | 6.3 |

There was no activation of the protease by these peptides at any of the concentrations tested. In fact there was a slight inhibition of the protease by these peptides. These results indicate that these peptides represent a minimal domain of NS4A that binds to NS3, but does not activate the enzyme and can, in fact, lower/inhibit the basal activity of NS3. In combination with the mutagenesis results, it is clear that these compounds, derivative compounds and the like are agents that inhibit the activity of NS3 by binding in the NS4A binding site.

In addition the NS4A binding site is close to the substrate binding site, and therefore two molecules, one that binds to the NS4A site and one that binds to the substrate binding site can be linked into one. The increase in the binding that is cause by the addition of a second site is equivalent to multiplying the binding constants. For example the Ka for the NS4A V23F peptide is 1×10⁻⁶ M and there is a substrate inhibitor with Ks of approx. 20×10⁻⁶M, the binding constant that would result from linking the two peptides is calculated to be 20×10⁻¹², which is a very strong binding constant.

The nature if the linker is also important. A linker consisting of 9 Arginine residues has been shown to facilitate the transport of whole proteins across membranes (Wender et al 2000 PNAS 97, 13003-13008), and can further serve to facilitate immune evasion because the enantiomers of natural amino acids are not immunogenic. It has been shown that 9 D-Arginine residues can also serve to facilitate the transport of peptides across membranes. Thus the unification of these two sites with a linker makes a compound that is non-immunogenic, can cross membranes and binds to and inhibits the HCV NS3 protease.

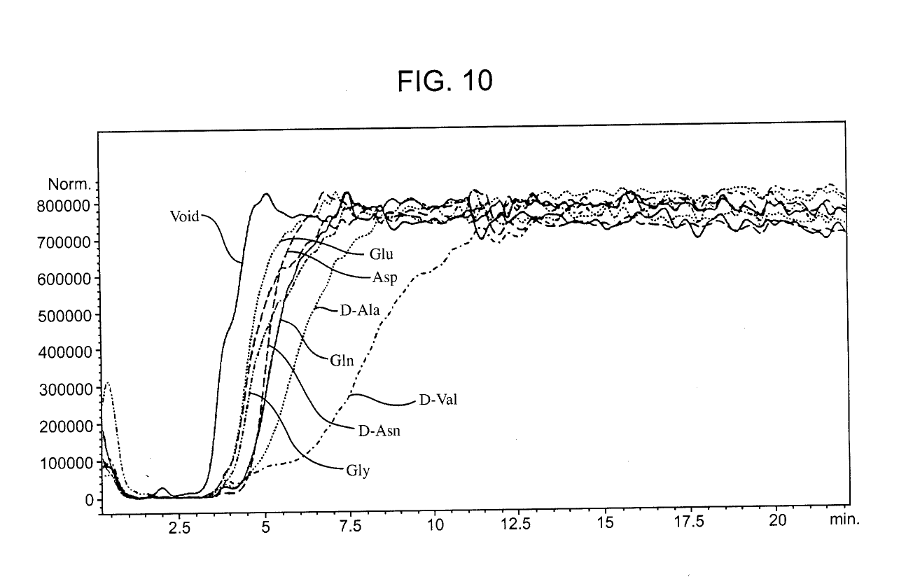

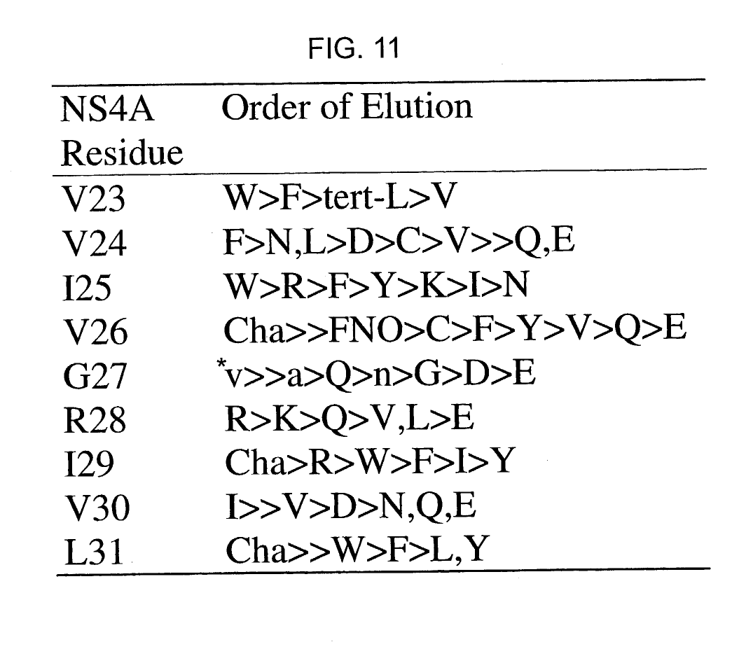

Libraries having varying residues for each residue of the longest of these peptides (VVIVGRIVL) were synthesized and the screened for binding to NS3 using FAC-MS. An exemplary chromatogram from the library with changes at residue glycine 27 is shown in FIG. 10 (VVIVXRIVL, where X was Glu, Asp, Gln, D-Ala, D-Asn, D-Val, or Gly). The results of the screening are shown qualitatively in the table of FIG. 11, with the varying residue indicated in the first column, and the order of elution of residues substituted at the varying residue shown in the second column. Lowercase letters represent the corresponding D-amino acid (e.g., v=D-Val; V=L-Val). Residues separated by commas gave approximately the same elution profile. The residues are ordered according to the time of elution, with the residue substitution providing the longest elution time on the left.

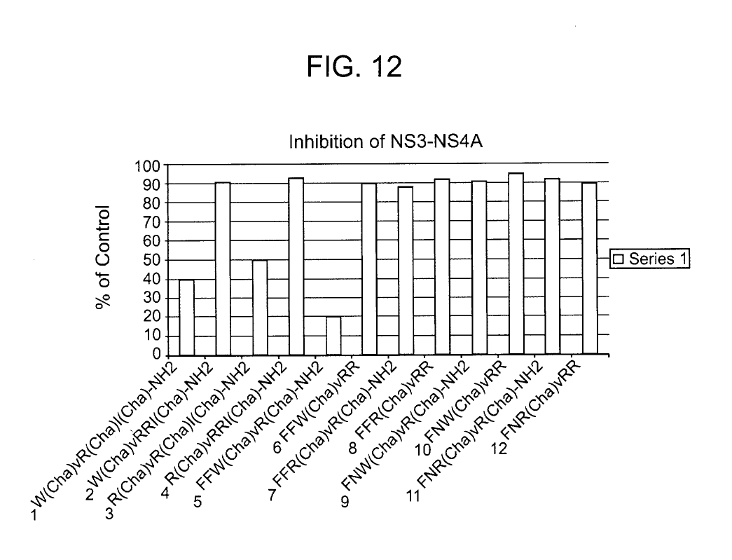

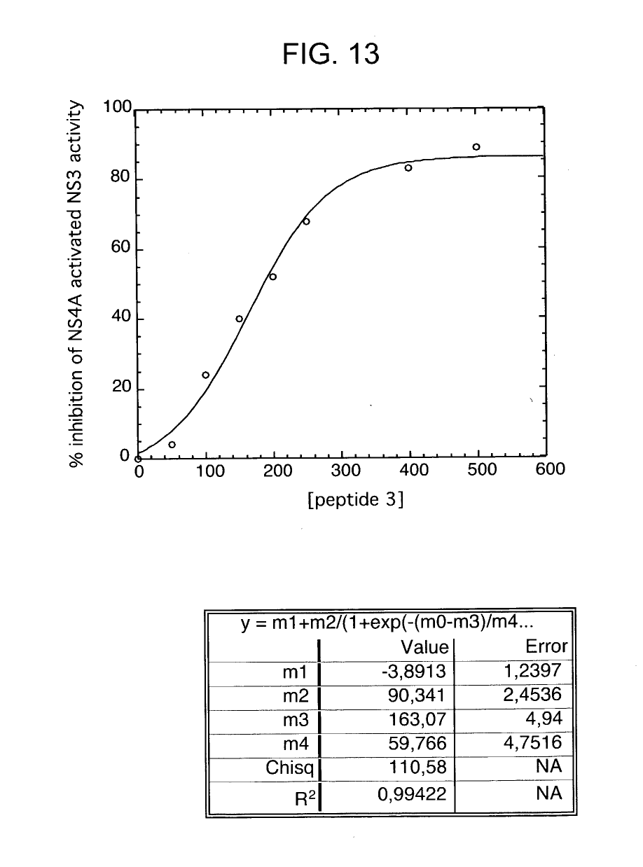

The results of the FAC-MS screening were used to design and synthesize several peptides whose sequences, the sequence of which are provided on the X-axis of FIG. 12. Again, lower case letters indicate the D-amino acid. Cha represents cyclohexylalanine. The peptides were assayed for inhibition of cleavage of 5A-pNA by NS3 and wt NS4A. The results are shown in FIG. 12. Three of these peptides inhibited this reaction peptide 1 (W(Cha)vR(Cha)I(Cha)-NH2); peptide 3 (R(Cha)vR(Cha)I(Cha)-NH2; and peptide 5 (FFW(Cha)vR(Cha)-NH2). Peptides 1 and 5 caused precipitation in the reaction mixture. Peptide 3 was used to determine an IC₅₀ value for the NS3/NS4A catalysed cleavage of 5A-pNA. The results are shown in FIG. 13, and the IC₅₀ was 163±5 μM.

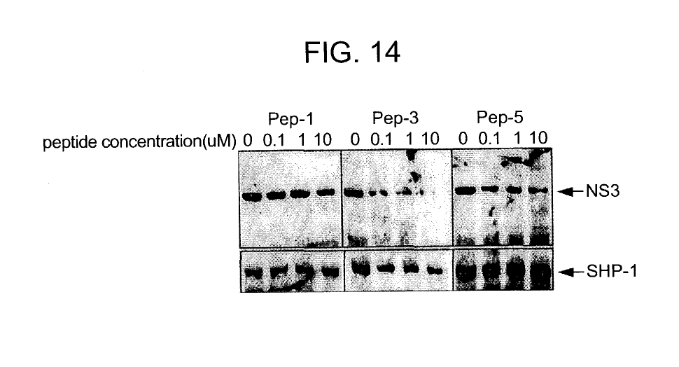

Peptides 1, 3, and 4 were also tested for their ability to inhibit the replication of HCV in tissue culture. Peripheral blood monocytic cells (PBMCs) were isolated from an HCV-infected patient, and induced to grow using PHA, IL-2 and IL-4. This induces production of HCV in these cells. The PBMCs were grown in the presence of varying concentrations of Peptides 1, 3, or 5 for 4 days, and then harveted, lysed, and analyzed by SDS-PAGE followed by Western blot using monoclonal antibodies that specifically bind HCV NS3 protease. The blots were then stripped and probed with antibodies for SHP-1 as a loading control. The results are shown in FIG. 14. These preliminary results (i.e., from a first experiment) indicate that peptide 3 inhibited replication of HCV in tissue culture.

That which is claimed is:

1. A peptide having the formula:
X₁-Z₁-Z₂-Z₃-Z₄-X₂
where X₁ is an integral number of residues from 0 to 5; Z₁ is a hydrophobic residue; Z₂ is a D-isomer of a hydrophobic residue; Z₃ is a positively charged residue; Z₄ is a hydrophobic residue or a positively charged residue; and X₂ is an integral number of residues from 0 to 3, wherein X₂ may optionally comprises a terminal amino group.

2. The peptide of claim 1, wherein Z₁ is M, F, W, or Cha.

3. The peptide of claim 1, wherein Z₂ is a D-amino acid of V, A, L, I, P, M, F, W, or Cha.

4. The peptide of claim 1, wherein Z₃ is K, R, or H.

5. The peptide of claim 1, wherein Z₄ is M, F, W, or Cha.

6. The peptide of claim 1, wherein Z₄ is a D-amino acid of V, A, L, I, P, M, F, W, or Cha.

7. A peptide having the formula:
X₁-Cha-vR-(Cha/R)-X₂
where X₁ is an integral number of residues from 0 to 5, X₂ is an integral number of residues from 0 to 3, Cha is cyclohexylalanine, v is D-valine, and wherein X₂ optionally comprises a terminal amino group.

8. The peptide of claim 7, wherein X₁ is W, R, FFR, FNW, FNR, or FFW.

9. The peptide of claim 7, wherein X₂ is a carboxyl group or I-Cha.

10. The peptide of claim 7, wherein Cha is β-cyclohexylalanine.

11. The peptide of claim 7, wherein the peptide has the formula W-Cha-v-R-Cha-I-Cha; R-Cha-v-R-Cha-I-Cha; F-F-W-Cha-v-R; W(Cha)vR(Cha)I(Cha)-NH2; R(Cha)vR(Cha)I(Cha)-NH2; and FFW(Cha)vR(Cha)-NH2.

12. The peptide of claim 7, wherein the peptide has the formula R-Cha-v-R-Cha-I-Cha.

13. A peptide having the formula:
X₁-GRI-X₂
where X₁ represents an integral number of residues from 0 to 9, and X₂ represents an integral number of residues from 0 to 10, with the proviso that the peptide is less than 13 residues.

14. The peptide of claim 13, wherein any of G, R, or I is a D-amino acid.

15. The peptide of claim 13, wherein G is substituted by a D-amino acid.

16. The peptide of claim 13, wherein X₁ is VV, VIV, IV, V, LVIV, t-LVIV, CVIV, PenVIV, FVIV, or an amino group.

17. The peptide of claim 13, wherein X₁ is C, Q, E, F, ChA, W, R, or Y, or a D isomer thereof.

18. The peptide of claim 13, wherein X₂ is a carboxyl group, V, VL, D-val, (D-val)L, V(D-leu) or (D-val)(D-leu).

19. The peptide of claim 13, where the peptide is of the formula VVIVGRI, VIVGRIVL, IVGRIVL, VGRIVL or GRIVL.

20. The peptide of claim 13, wherein one or more of the residues of the peptide are a D-amino acid.

21. A peptide having the formula:
X₁-DEMEE-X₂
wherein X₁ represents an acetyl group or an integral number of residues from 0 to 7, and X₂ is an acidic amino acid, with the proviso that the peptide is less than 13 residues in length.

22. The peptide of claim 21, wherein X₂ is Asp or Glu.

23. The peptide of claim 21, wherein the peptide has the formula:
X₁-DEMEEX₂-X₃
wherein X₁ represents an acetyl group or an integral number of residues from 0 to 7, and X₂ is an acidic amino acid, and X₃ represents an integral number of residues from 0 to 7, with the proviso that the peptide is less than 13 residues in length.

24. The peptide of claim 21, wherein the peptide has a formula selected from the group consisting of DEMEED, DEMEEE, Ac-DEMEED-OH and Ac-DEMEEE-OH.

25. A compound having the formula:
Peptide₁-L-Peptide₂
where Peptide₁ is a peptide of claims 1, 7, 13, or 21;
Peptide₂ is a peptide of claims 1, 7, 13 or 21; and
L represents a linker that links Peptides 1 and 2.

26. The compound of claim 25, wherein Peptide₁ is a peptide of claim 1 and Peptide₂ is a peptide of claim 14.

27. The compound of claim 25, wherein the linker is an amino acid sequence comprising polar amino acid residues.

28. The compound of claim 25, wherein the linker comprises D-arginine.

29. A pharmaceutical composition comprising a peptide of claim 1, 7, 13, 21, or 25 or a compound of claim 25.

30. A method for inhibiting replication of hepatitis C virus (HCV) in a subject having an HCV infection, the method comprising:
administering to the subject a peptide of claim 1, 7, 13, or 21, or a compound of claim 25, in an amount effective to inhibit HCV replication in the subject.

31. A method for reducing viral load of hepatitis C virus (HCV) in a subject having an HCV infection, the method comprising:
administering to the subject a peptide of claim 1, 7, 13, or 21 or a compound of claim 25, in an amount effective to reduce viral load in the subject.
